(19) United States
(12) Patent Application Publication
(10) Pub. No.: US 2005/0227266 A1
(43) Pub. Date: Oct. 13, 2005

(54) Biomarker:compound correlations in cancer diagnosis and therapy

(76) Inventors: Douglas T. Ross, Burlingame, CA (US); Brian Z. Ring, Foster City, CA (US); Robert S. Seitz, Hampton Cove, AL (US)

Correspondence Address: CHOATE, HALL & STEWART LLP, TWO INTERNATIONAL PLACE, BOSTON, MA, 02110, US

(21) Appl. No.: 11/050,567

(22) Filed: Feb. 3, 2005

Related U.S. Application Data
(60) Provisional application No. 60/542,370, filed on Feb. 6, 2004.

(51) Int. Cl. C12Q 1/68; G01N 33/574

(52) U.S. Cl. 435/6; 435/7.23

(57) ABSTRACT

The present invention provides methods and reagents relating to establishing and using biomarker:chemical compound correlations. The invention provides correlated biomarker:compound pairs, and further provides methods of using such pairs, for example, in the identification of tumors or tumor cells likely to be responsive or resistant to particular therapy, and/or the identification of chemical compounds likely (or unlikely) to be useful in the treatment of particular tumors.

### PRIORITY INFORMATION

The present application claims priority to provisional application U.S. Ser. No. 60/542,370 filed Feb. 6, 2004 the entire contents of which are hereby incorporated by reference.

### BACKGROUND OF THE INVENTION

A major challenge of cancer treatment is to identify or select a therapeutic regimen that maximizes efficacy and minimizes toxicity for a given patient. Different tumors of the same type can respond very differently to particular therapeutic agents. Moreover, for many tumors, there are no available effective therapies.

In some cases, it has been possible to identify a detectable marker that can classify tumors into those that are or are not likely to respond to a particular agent. For example, one factor considered in prognosis and in treatment decisions for breast cancer is the presence or absence of the estrogen receptor (ER) in tumor samples. ER-positive breast cancers typically respond much more readily to hormonal therapies such as tamoxifen, which acts as an anti-estrogen in breast tissue, than ER-negative tumors.

Though useful, these analyses only in part predict the clinical behavior of breast tumors. There is phenotypic diversity present in cancers that current diagnostic tools fail to detect. As a consequence, there is still much controversy over how to stratify patients amongst potential treatments in order to optimize outcome (e.g., for breast cancer see “NIH Consensus Development Conference Statement: Adjuvant Therapy for Breast Cancer, Nov. 1-3, 2000”, J. Nat. Cancer Inst. Monographs, 30:5-15, 2001 and “Predictive molecular markers in the adjuvant therapy of breast cancer: state of the art in the year 2002”, Di Leo et al., Int. J. Clin. Oncol. 7:245-253, 2002).

There clearly exists a need for improved methods and reagents for classifying tumors as likely or unlikely to respond to therapy with particular agents. There is an additional need to identify detectable biomarkers in or on tumors whose presence or absence correlates with their expected responsiveness. There is a further need to identify additional cytotoxic agents useful in the treatment of particular tumors.

### SUMMARY OF THE INVENTION

The present invention provides tools, strategies, information, and reagents that allow researchers to define biomarkers by establishing correlations between biological agents present or active in or on tumor cells and responsiveness to anti-tumor treatments. The invention also provides correlated biomarker:chemical compound pairs that are useful, for example, in the identification of tumors likely to be responsive or resistant to particular therapy (e.g., to treatment with a particular chemical compound), and/or the identification of chemical compounds likely to be useful in the treatment of particular tumors.

In one aspect, the invention provides systems for defining biomarker:chemical compound correlations, and further provides correlated biomarker:chemical compound pairs.

In another aspect, the invention provides methods of classifying tumors (or tumor cells) as likely or unlikely to respond to therapy with a chemical compound, for example by providing or obtaining a tumor sample from a patient; detecting in the tumor sample a correlated biomarker characterized in that presence or absence of the biomarker has been correlated with responsiveness or lack of responsiveness to a selected chemical compound; and classifying the tumor as likely or unlikely to respond to the chemical compound based on the results of the detection step.

In yet another aspect, the invention provides methods of identifying biomarkers that are predictive of tumor responsiveness to particular chemical compounds by providing an expression or activity dataset for a predetermined collection of tumor cells; providing a chemical compound toxicity dataset for the tumor cells; and establishing a correlation between expression of at least one biomarker and toxicity of at least one compound such that expression of the at least one biomarker is predictive of tumor responsiveness to the at least one compound.

In still another aspect, the invention provides a method of identifying chemical compounds whose ability to inhibit tumor cell growth correlates with expression of a particular biomarker by providing an expression or activity dataset for a predetermined collection of tumor cells; providing a chemical compound toxicity dataset for the tumor cells; and establishing a correlation between expression of at least one biomarker and toxicity of at least one compound such that the compound is predicted to effectively inhibit growth of tumor cells expressing the biomarker.

The invention additionally provides methods of identifying lead compounds for use in treating cancer by establishing correlations between biomarkers and chemical compounds. These compounds may prove leads for identification of related therapeutic agents, or may be therapeutic agents themselves. In particular, the invention provides methods for detecting in a tumor sample from a patient a correlated biomarker presented in FIG. 4; and administering to the patient a therapeutic agent comprising the appropriate correlated chemical compound listed in FIG. 4.

### BRIEF DESCRIPTION OF THE DRAWING

FIG. 1. FIG. 1A displays hierarchical cluster analysis of a gene expression database including data for approximately 17,000 genes across the NC160 cell line panel. Red denotes a high level of expression; green denotes a low level of expression. FIG. 1B displays hierarchical cluster analysis of the GI50 database of compound sensitivity of the NCI60 cell line panel. Violet reflects a high GI50; blue depicts a low GI50. In both Panels (i.e., FIG. 1A and FIG. 1B), cell lines are color coded to reflect their tissue of origin (blue=lung; red=hematapoietic; orange=ovarian; pink=breast; green=colon; yellow=renal; brown=melanoma; black=CNS; purple=prostate).

FIG. 2. FIG. 2 displays a comparison of the normalized pattern of erbB2 gene expression with the normalized GI50 values for compound NSC # 683039 across the NCI60 cell lines.

FIG. 3. FIG. 3 displays a comparison of the normalized pattern of PGP gene expression with the normalized GI50 values for Paclitaxol across the NCI60 cell lines.

FIG. 4. FIG. 4 is a Table of biomarker:compound correlates that reflect and/or predict sensitivity or resistance of tumor cells expressing the biomarker to growth inhibition by the compound. The pairs presented in FIG. 4 are positively correlated.

FIG. 5. FIG. 5 is a Table of biomarker:compound correlates that reflect and/or predict sensitivity or resistance of tumor cells expressing the biomarker to growth inhibition by the compound. The pairs presented in FIG. 5 are negatively correlated.

FIG. 6. FIG. 6 is a Table that lists a preferred subset of the positively correlated biomarker:compound pairs of FIG. 4.

### DEFINITIONS

Antibody. In general, the term “antibody” refers to an immunoglobulin, which may be natural or wholly or partially synthetically produced in various embodiments of the invention. An antibody may be derived from natural sources (e.g., purified from a rodent, rabbit, chicken (or egg) from an animal that has been immunized with an antigen or a construct that encodes the antigen) partly or wholly synthetically produced. An antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, 2:750-765, 2002, and references therein. Preferred antibodies, antibody fragments, and/or protein domains comprising an antigen binding site may be generated and/or selected in vitro, e.g., using techniques such as phage display (Winter, G. et al., Annu. Rev. Immunol. 12:433-455, 1994), ribosome display (Hanes, J., and Pluckthun, A. Proc. Natl. Acad. Sci. USA. 94:4937-4942, 1997), etc. In various embodiments of the invention the antibody is a “humanized” antibody in which for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., Nature Biotechnology, 16:535-539, 1998. An antibody may be polyclonal or monoclonal, though for purposes of the present invention monoclonal antibodies are generally preferred.

Candidate biomarker. A “candidate biomarker”, as that term is used herein, is any detectable entity that is expressed by, present in or on, or active in or on a tumor cell. In many preferred embodiments, a candidate biomarker is a gene or protein that is expressed or active in or on a tumor cell. A candidate biomarker may be detected through detection of the candidate biomarker itself, or by detection of any other product or component whose presence or level is indicative of presence or activity of the candidate biomarker. For example, where the candidate biomarker is a protein, it may be detected directly (e.g., using an antibody or other ligand that interacts specifically with the candidate biomarker) or the candidate biomarker may be detected by detecting expression of its gene. In some instances, a candidate biomarker may have a biological activity (e.g., an enzymatic activity), and may be detectable through analysis of a reagent or product that it affects, utilizes, or produces.

Correlated biomarker: A “correlated biomarker”, as that term is used herein, refers to a compound, complex, or entity, whose presence or level in a cell or tumor sample, is correlated with a biological event or clinical outcome. For example, the estrogen receptor is a correlated biomarker for breast cancers that are likely to respond to hormonal therapy. Where the term “biomarker” is used without the qualifier “candidate” or “correlated”, those of ordinary skill in the art will appreciate that, consistent with its typical usage in the art, the term generally refers to a correlated biomarker.

Chemical compound. In general, the terms “chemical compound” or “compound” are used herein to refer to any agent or entity that can be used as a chemotherapeutic agent to inhibit the growth or viability of tumor cells. Specifically, agents that can kill or inhibit the growth of cells are included. The term is not intended to be limited to any particular class of chemical entities, but preferred agents include small molecules. Alternative preferred agents include antibodies, or antibody fusions (e.g., antibodies or antibody fragments linked to a toxin).

Dataset. A “dataset” according to the present invention, is a collection of individual data points of the same type. For instance, an “expression/activity dataset” is a collection of data points representative of expression or activity levels of individual genes or gene products expressed in or by tumor cells. Comparably, a “chemical compound dataset” is a collection of data points representative of the effect of individual chemical compounds on the growth or viability of tumor cells. It will be appreciated that preferred datasets generally contain relative data points. For example, a preferred expression/activity dataset will contain relative expression or activity levels of individual genes or gene products expressed in or by tumor cells, and a preferred chemical compound dataset will contain data points representative of the relative effect of the individual chemical compounds on the growth or viability of tumor cells.

High quality dataset. The phrase “high quality dataset” is used herein to refer to a dataset that is removed from an original “raw” dataset by the application of at least one filter to the raw dataset.

Isolated. The term “isolated”, as used herein refers to a chemical or biological entity that 1) does not exist in nature; 2) is produced or purified through a process that requires the hand of man; 3) is separated from at least some of the components with which it is associated in nature; and/or 4) is separated from at least some of the components with which it is associated when originally produced.

Raw dataset. The phrase “raw dataset” is used herein to refer to a set of data generated directly by an experiment or obtained from a public source.

Small molecule. Small molecule is a term of art that is applied to organic compounds, typically having a molecular weight of less than 1500. Small molecules may be naturally-occurring compounds or chemically synthesized or prepared compounds. In certain embodiments, the term is applied preferentially to non-polymeric compounds (e.g., non-peptide, non-nucleic acid).

### DETAILED DESCRIPTION OF CERTAIN PREFERRED EMBODIMENTS OF THE INVENTION

As discussed above, the present invention provides correlated pairs of tumor biomarkers and potential or known chemotherapeutic agents, as well as methods, information, and reagents that allow such pairs to be defined and/or used.

Establishing Biomarker: Compound Correlations

In general, according to the present invention, correlated pairs of tumor biomarkers and chemical compounds that are or could be used as chemotherapeutic agents are established by statistical comparison of an expression/activity dataset with a chemical compound dataset.

Expresssion/Activity Dataset

An “expression dataset” or an “activity dataset” is a collection of data points that represent the expression or activity level of individual candidate biomarkers (e.g., genes or proteins expressed in or by tumor cells). For the purposes of the present invention, the precise nature of the data points is not important so long as expression or activity is quantified.

For example, an expression/activity dataset may contain mRNA data points that quantify any step in candidate biomarker expression or activity including, for example, transcription, post-transcriptional processing, splicing, translation, post-translational modification, folding, subcellular localization, binding or enzymatic activity, etc. In certain preferred embodiments of the invention, the data points in the expression/activity dataset are mRNA levels. In other preferred embodiments, the data points in the expression/activity dataset are antibody binding levels, for example representing antibody binding to cell surface or other markers.

According to the present invention, it will often be desirable to assemble expression/activity datasets containing information on the largest possible number of different candidate biomarkers across the largest possible number of tumor cells. However, as will be appreciated by those of ordinary skill in the art, it will also often be desirable to ensure that the data points are obtained under experimental conditions that are as closely comparable as possible, in order to minimize variations due to experimental conditions rather than to tumor type.

Expression/activity datasets may be assembled from data points that represent the expression or activity of candidate biomarkers in isolated tumor cells or cell lines, or in more complex tumor samples. For example, U.S. Ser. No. 10/915,059 and PCT Serial No. US 04/26005 (which both claim priority to U.S. Ser. No. 60/494,334, filed Aug. 11, 2003) both incorporated herein by reference, describe methods that may be utilized to prepare standardized tumor samples for analysis by antibody binding to cell surface markers.

As described below in the Examples, the present inventors have obtained mRNA samples from a set of standard tumor cell lines assembled by the National Cancer Institute. To the extent possible, cells were grown under comparable conditions, mRNA was isolated, and the mRNA was hybridized to a microarray containing more than 44,000 ESTs. Those of ordinary skill in the art will appreciate that microarrays provide an attractive platform for the simultaneous analysis of the expression levels of multiple genes and/or gene products (e.g., alternative splice variants). Microarrays may be purchased from any of a variety of commercial sources (e.g., Agilent Technologies, Affymetrix, Inc., etc.), or may be prepared by the researchers.

In general, larger numbers of ESTs on such microarrays allow simultaneous analysis of larger numbers of genes or gene products. Preferably, ESTs representing at least ten thousand genes are included on microarrays utilized in accordance with the present invention; more preferably at least about 15, 20, 25, 30, 35, 40, 45, 50, 55, 60, 65, 70, 75, 80, 85, 90, 95, or 100 thousand genes are represented on individual microarrays.

Of course, mRNA expression levels need not be determined by microarray analysis, but instead can be established by any available experimental analysis or indeed without any experimental analysis (e.g., by consulting published or other reports of gene expression levels under particular conditions). On the other hand, it will often be desirable to assemble datasets of data points that were collected with great precision and limited noise, in order to increase the probability that defined correlations will be based on “true” variations in gene expression rather than extraneous noise in the measurements. Accordingly, in particularly preferred embodiments of the present invention, expression datasets are established using data points from two-color hybridization of gene expression microarrays, as this strategy has displayed excellent reproducibility and precision of measurement (see, for example, Ross et al., Nat Genet. 24:227, 2000; Perou et al., Nature 406:747, 2000).

Once expression (or activity) level data points have been collected, any of a variety of filters may be applied to exclude data points that might be unreliable or generate correlations not reflective of biological interactions or effects. For example, as described in the Examples, the present inventors have assembled “high quality” expression datasets by excluding one or more of: 1) data points from genes that were undetectable in a meaningful fraction (e.g., greater than 5%, 10%, 15%, 20%, 25%, etc.) of cell lines; 2) data points from leukemia cell lines; 3) data points for which kurtosis was too high (e.g., greater than about 50, 30, 15, 10, 5, etc.); 4) data points reflecting broad physiological characteristics (e.g., experimental conditions) that varied across the cell lines; 5) data points that showed insignificant variance (e.g., standard deviations less than about 0.5, 1, 2, etc.).

As illustrated in the Examples, the exclusion of data points for genes that were not detectable in at least 80% of cells excluded information obtained with 27,000 ESTs, so that “high quality” data were obtained for only 17,000 genes. In preferred embodiments of the invention, “high quality” data are obtained for at least 10, 15, 20, 25, 30, 35, 40, 45, 50, 55, 60, 65, 70, 75, 80, 85, 90, 95, or more than 100 thousand genes or gene products.

Those of ordinary skill in the art will readily appreciate that the present invention is not limited to the application of any particular filter or filter set to an expression/activity dataset. No filtering is required for the practice of the present invention. However, the present inventors have found that application of one or more exclusionary filters can improve the quality of the dataset and can increase significantly the reliability of correlations observed in the database comparison.

Compound Toxicity Dataset

A compound toxicity dataset is a collection of data points that represent the inhibitory activity of individual chemical compounds or agents on tumor cell growth or viability.

A compound toxicity dataset may be assembled by testing individual compounds against different tumor cell samples (e.g., cell lines or other tumor samples), or may be assembled from published or other reports. As with the expression/activity dataset, it is generally desirable to include data points collected under conditions as comparable as possible.

Any chemical compound may be tested or analyzed according to the present invention for its toxicity to tumor cells. A large number of different compounds are known to have cytotoxic effects and/or to be useful in the inhibition of tumor cell growth. Particularly, preferred compounds for use in accordance with the present invention are small molecules, which may be natural, synthetic, or semi-synthetic compounds, and antibodies or other specific ligands of tumor biomarkers.

Particularly preferred chemical compounds for use in accordance with the present invention are those that have undergone clinical testing in humans and have a known, acceptable toxilogical profile. Particularly preferred compounds are approved for use in cancer patients.

In some embodiments of the present invention, it may be desirable to assemble compound toxicity datasets from data points for a collection of chemical compounds that share one or more common structural features. In other embodiments, diversity of chemical structure may be preferred, or may be irrelevant.

As with the expression/activity dataset, the nature of the data points that make up the chemical compound dataset is not intended to limit the scope of the present invention. For example, the data points may be concentrations at which the compounds inhibit cell growth by a particular amount at a particular time, or may be expression levels of a reporter or other gene whose levels reflect cell viability status. Alternatively or additionally, the data points could be amounts of labeled nucleotides (e.g., radionuclides, BrdU, etc.) incorporated into dividing cells, amounts of formazan product produced (in an MTT assay, indicating reduction of MTT by living but not dead cells), levels of staining with antibodies to a cells proliferation marker (e.g., KI67, PCNA, etc.), levels of DNA fragmentation (e.g., detected via a TUNEL assay and indicative of apoptosis), levels of caspase activity (indicative of apoptosis), levels of LDH or chromium 51 released from lysed cells, etc. Those of ordinary skill in the art will readily appreciate that the data points can be any quantitative or qualitative representation of cell viability.

Since 1990, the Developmental Therapeutics Program (“DTP”) at the National Cancer Institute (“NCI”) has performed in vitro testing of known or putative cytotoxic compounds in the NCI60 cell lines (see, for example, Alley et al., Cancer Res. 48:589, 1988; Grever et al., Seminars in Oncology 19:622, 1992; Boyd et al., Drug Dev. Res. 34:91, 1995, each of which is incorporated herein by reference). Compounds are tested according to a standard protocol (dtp.nci.nih.gov/branches/btb/ivclsp.html, reproduced as Appendix B), and GI50 data (i.e., data indicating the concentration of a compound that inhibits growth of a particular cell line by 50% after 48 hours of exposure) for many compounds, including a set of 171 so-called Standard Agents (dtp.nci.nih.gov/docs/cancer/searches/standard_agent.html, reproduced as Appendix C) are made publicly available. Sets of DTP data points are useful compound toxicity datasets for use in the practice of the present invention.

As with the expression/activity dataset, it will sometimes be desirable to apply one or more filters to the chemical compound toxicity dataset to achieve a “high quality” dataset. For example, as described in the Examples, the present inventors have prepared a “high quality” dataset from the DTP dataset by excluding 1) data points for compounds for which measurements had not been obtained for a significant fraction (e.g., 5%, 10%, 15%, 20%, 25%, 30%, etc.) of the cell lines; 2) data points for compounds that showed little or no toxicity across the cell lines; 3) data points for compounds with cytotoxicity that varied minimally around a large concentration (e.g., standard deviations in GI50 of less than about 0.5, 1, 2, etc.); 4) data points for which the spread of the data (e.g., the maximum GI50 value minus the minimum GI50 value) was less than the average variation found within repeat measurements of the cytotoxicity within each cell line for that compound; and/or 5) compounds for which the kurtosis was too high (e.g., kurtosis in GI50 greater than about 50, 30, 15, 10, 5, etc.).

It will be appreciated by those of ordinary skill in the art that such filtering is not required for the practice of the present invention, but may be useful to ensure the reliability of detected correlations. Also, when filters are applied, they are not limited to those specifically described herein. Filters may be applied before or after establishment of a correlation.

Comparing Datasets

Inventive biomarker:compound correlations are established by comparing the expression/activity expression and compound toxicity datasets. Generally, comparisons are preferably performed between every candidate biomarker in the expression/activity dataset, and every record of cytotoxicity in the compound dataset. Such comparisons can quantify the association between the variables, e.g., using a Pearson or Spearman correlation. Alternatively or additionally, a statistical test (e.g., Student's T or Mann-Whitney test) may be used to measure the significance of the comparison. Also, before comparisons are performed, it may be desirable to modify the measurements in one of a variety of ways. For example, to aid in the identification of non-linear relationships between compound toxicity and presence of a biomarker (e.g., a threshold response), values above or below a certain limit may be given a common value, or weighted, before the comparison is measured.

Cell lines that lack data for either expression/activity or toxicity are preferably not used in the correlation measurement. Where the absolute value of the correlation is above a certain limit (e.g., 0.2, 0.3, 0.4, 0.5, etc.), the correlation is retained, as is the number of contributing cell lines. In general, the quality of a correlation is assessed by any of a number or combination of possible filters. These may include, but are not limited to, for example:
1) comparing correlations where certain cell lines are removed (e.g., leukemia cell lines).
2) requiring that a minimum number of cell lines contribute to the correlation (e.g., greater than 50%, 60%, 70%, 80%, etc.).
3) excluding correlations with compounds for which the kurtosis in the GI50 scores is too high (e.g., greater than 50, 30, 15, 10, 5, etc.).

Also, correlated pairs may be prioritized through the application of one or a combination of selection rules such as, for example:
1) the strength of the correlation between the candidate biomarker and the compound cytotoxicity measurement (e.g., greater than 0.5, 0.6, 0.7, etc.).
2) the pattern of expression of the candidate biomarker can be considered for its applicability as a potential pharmacological target (e.g., normal tissue expression, tumor tissue expression, etc.).
3) a minimal potency of the compound as measured by the GI50 assessment of cytotoxicity (e.g., GI50 at greater than 1X10⁻⁵ M, 1⁻⁶ M, 10⁻⁷ M, etc.).
4) a minimal level of expression of the candidate biomarker.
5) prior information regarding the potential of the candidate biomarker to serve as a pharmacological target for cancer (e.g., a target with biologic activity consistent with past successful small molecule development for cancer treatment including, for example, kinases, phosphatases, or other enzymes or proteins with defined biochemical functions).
6) prior information regarding the applicability of the compound for pharmacological modification for drugability.
7) the uniqueness of the compound GI50 pattern amongst the measured compounds in the cytotoxicity dataset.
8) the uniqueness of candidate biomarker's expression pattern in the expression dataset.
9) subjective assessment of the pattern of variation of either the compound or expression data across the measured cell lines.

Those of ordinary skill in the art will appreciate that the strategies described herein define biomarker:compound correlations that can predict the likelihood that a particular tumor will be sensitive or resistant to a selected therapy, and that can identify new candidate therapeutic agents. Those of ordinary skill in the art will further understand that the information provided by the present invention is not limited to establishing that the presence of a particular biomarker candidate indicates increased sensitivity to a given compound, but rather can encompass more subtle relationships. For example, the present invention can establish correlations between, for example, a particular level of candidate biomarker expression and sensitivity to a designated amount of chemical compound. The information does not necessarily dictate the manner in which the biomarker will be assessed in patient clinical material. For instance, in some cases the mere detectable presence of a biomarker may not be predictive of a particular outcome, but presence at a level above a designated amount may be. An example of this is outlined in Example 2 where a known biomarker for a current therapeutic is re-discovered by the correlation between gene expression and compound sensitivity patterns, however, in clinical practice the measurement of the biomarker is assessed using a semi-quantitative measure that distinguishes negative, weak-positive, moderate-positive and strong-positive biomarker expression. In this case, it has been determined that treatment of patients with biomarker-directed compounds are only indicated in tumors where the biomarker is expressed at moderate or strong levels.

Correlated Biomarker:Compound Pairs

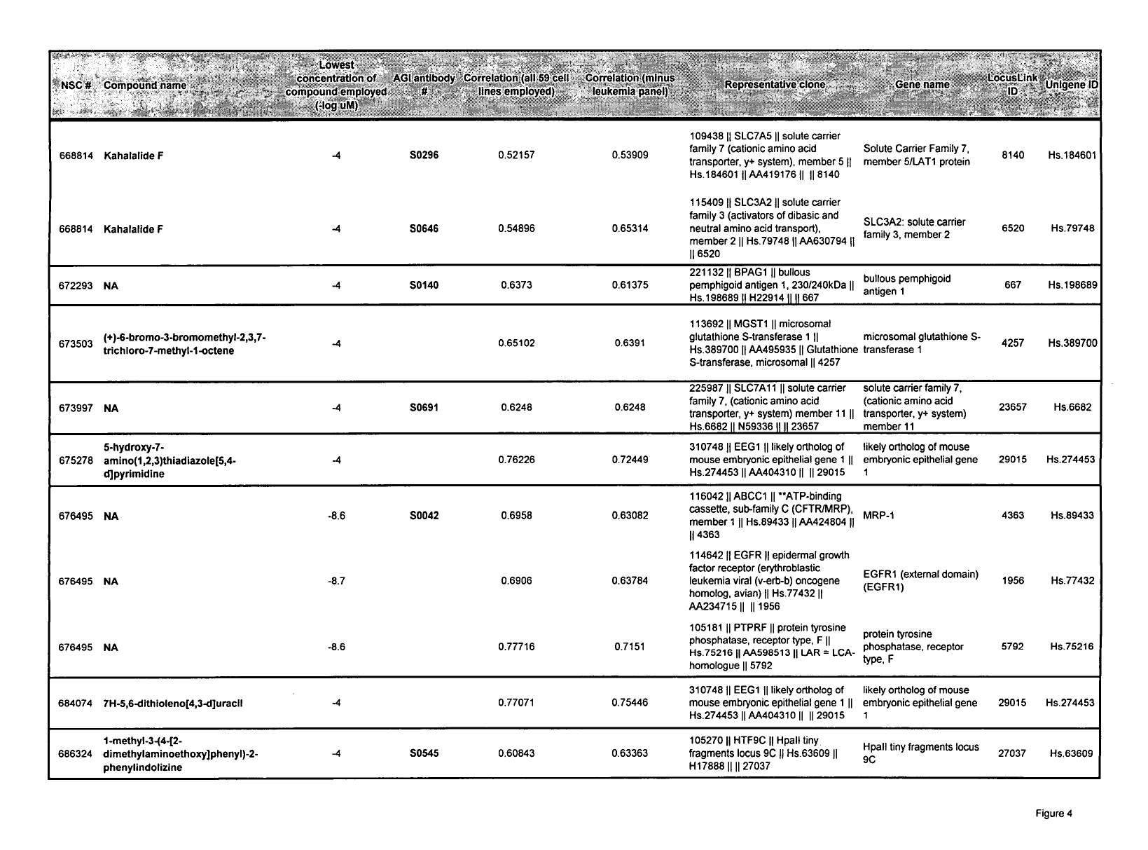

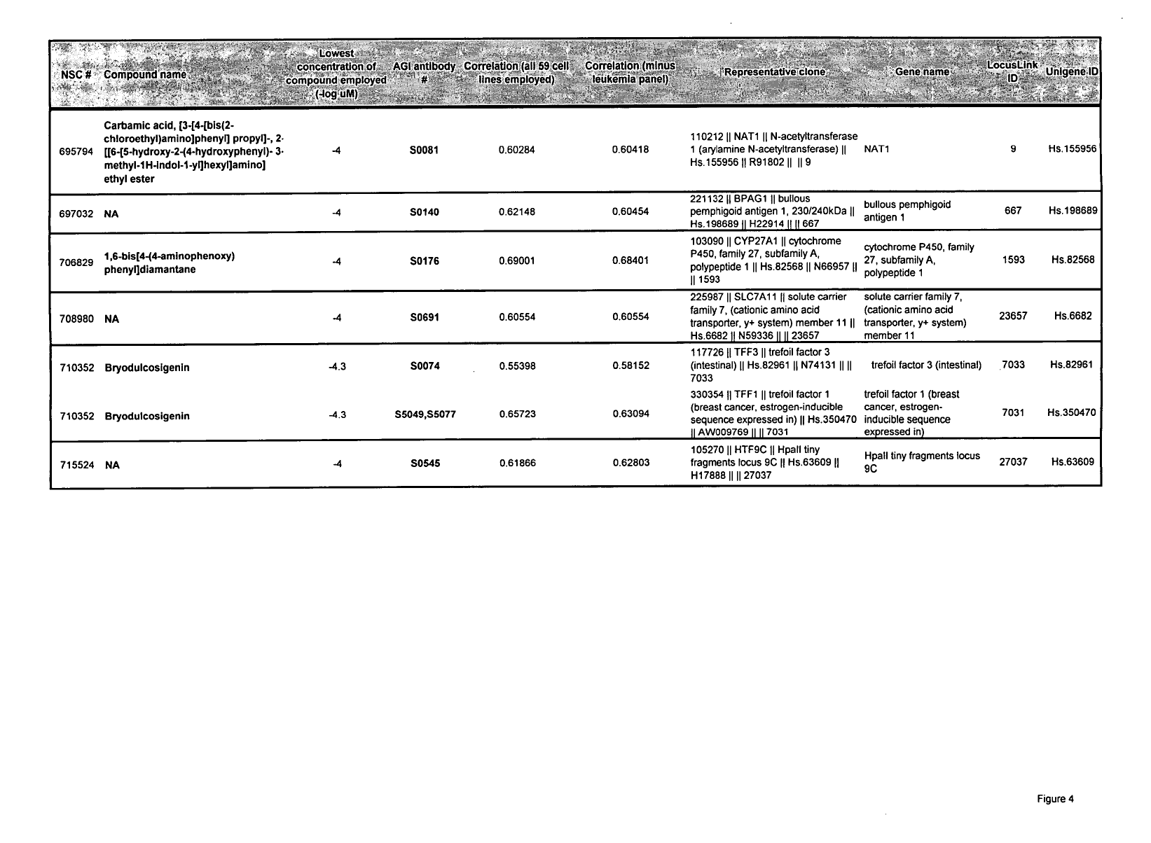

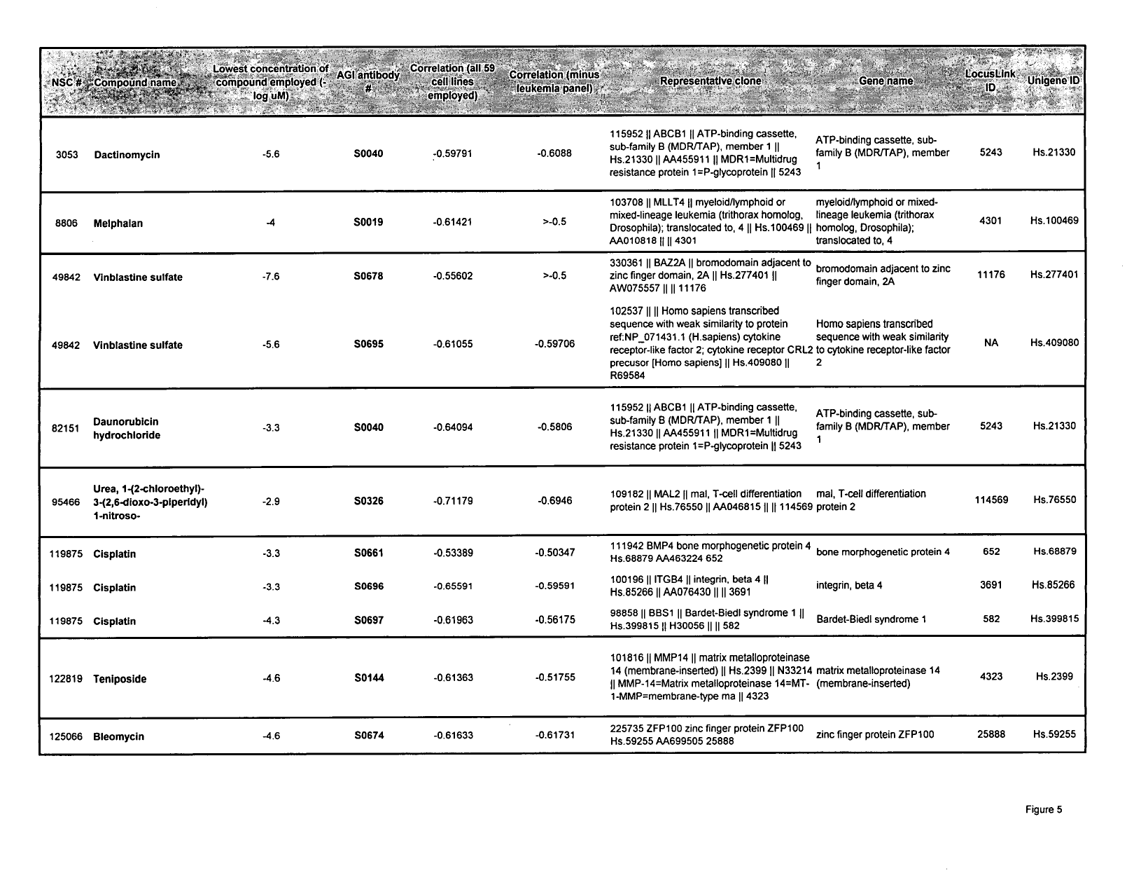

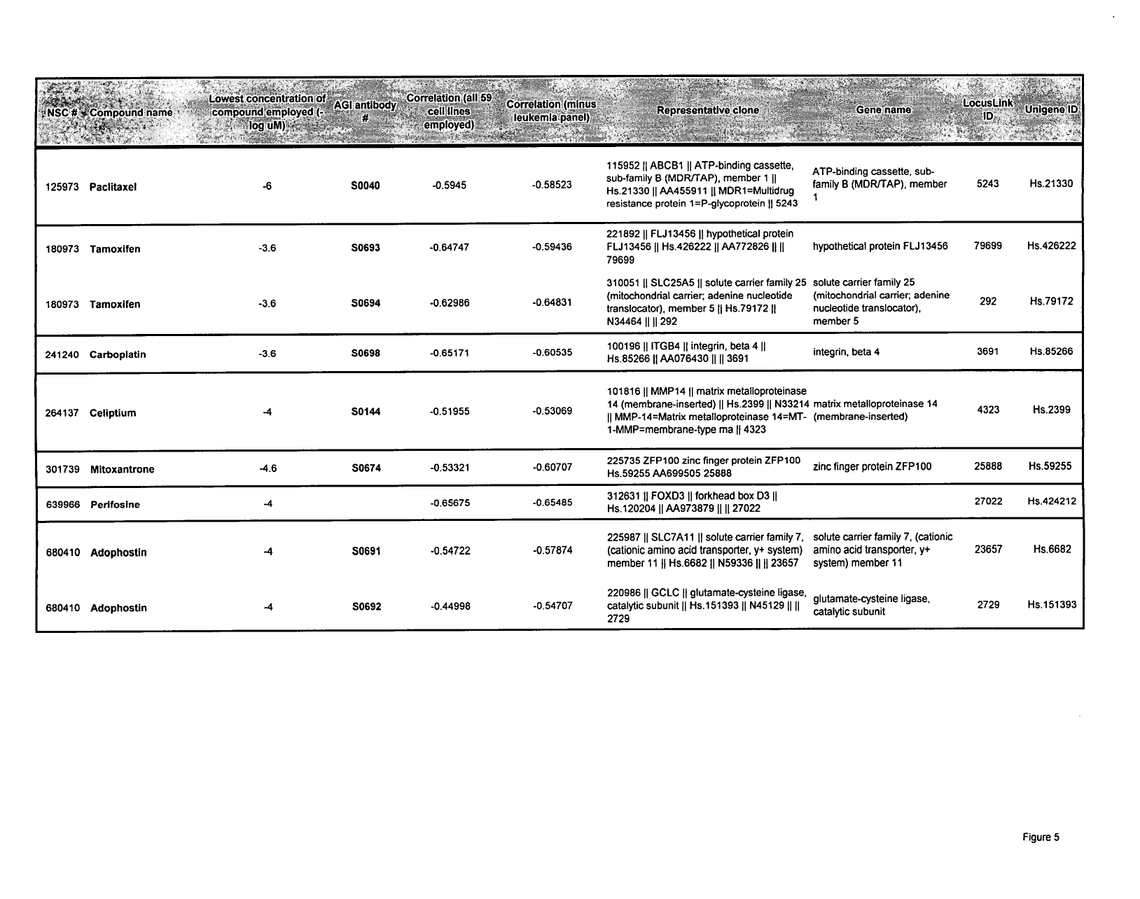

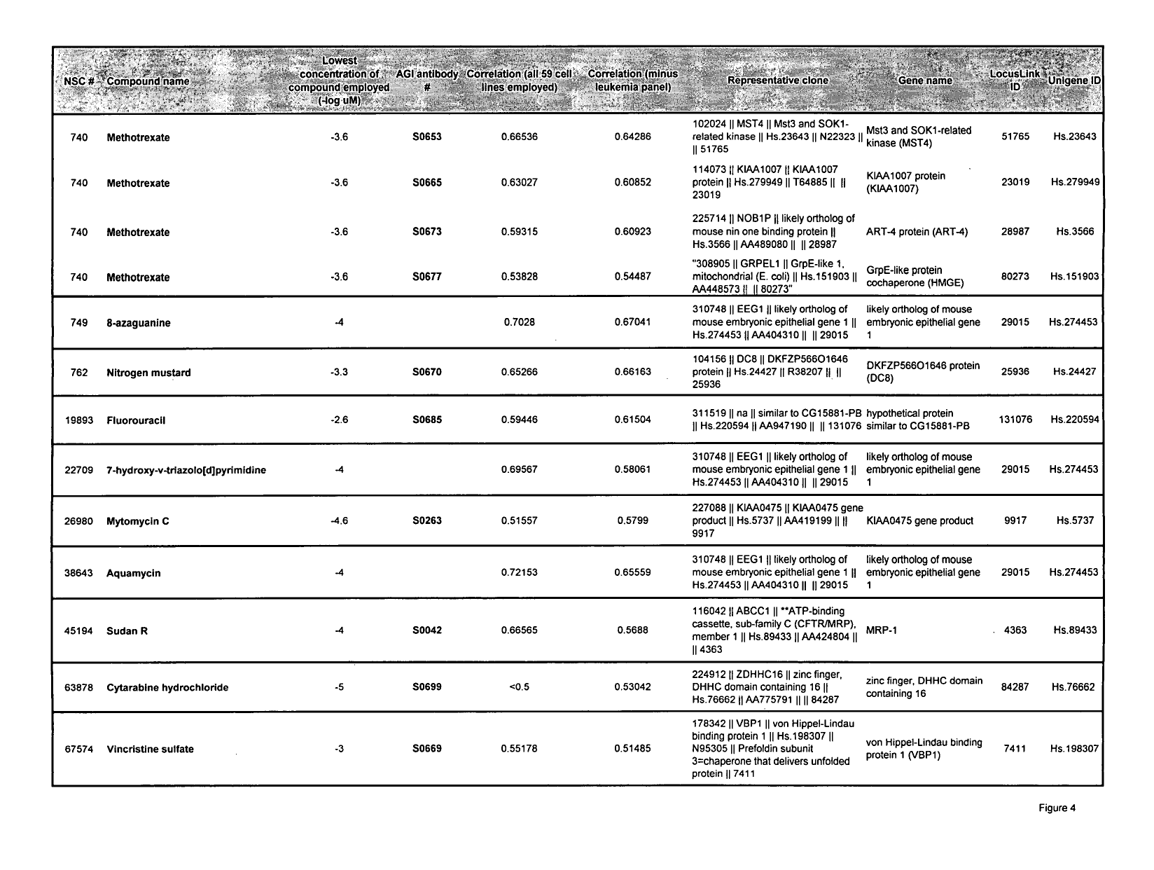

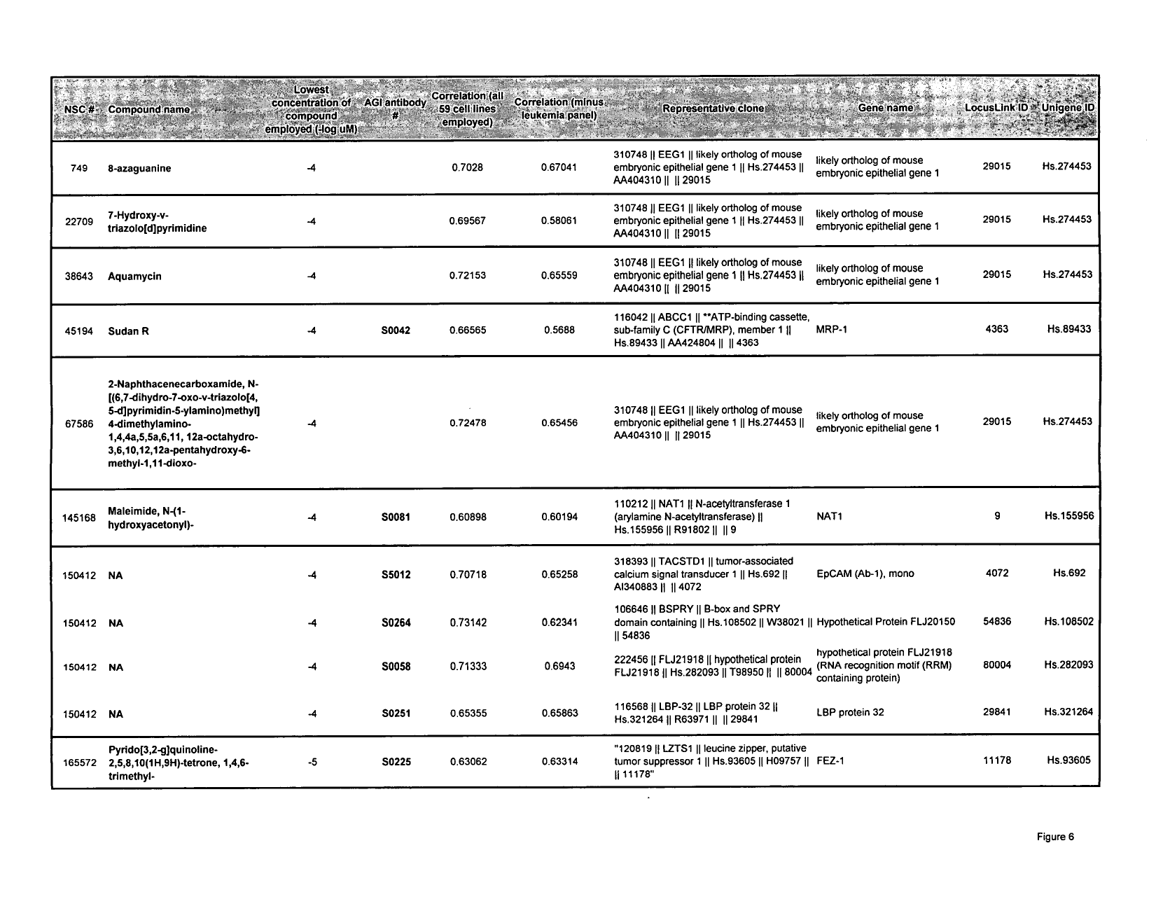

As set forth in the Examples, the present inventors have defined 72 biomarker:compound correlates that reflect and/or predict sensitivity or resistance of tumor cells expressing the biomarker to growth inhibition by the compound. FIG. 4 presents a Table of positively correlated pairs. FIG. 5 presents a Table of negatively correlated pairs.

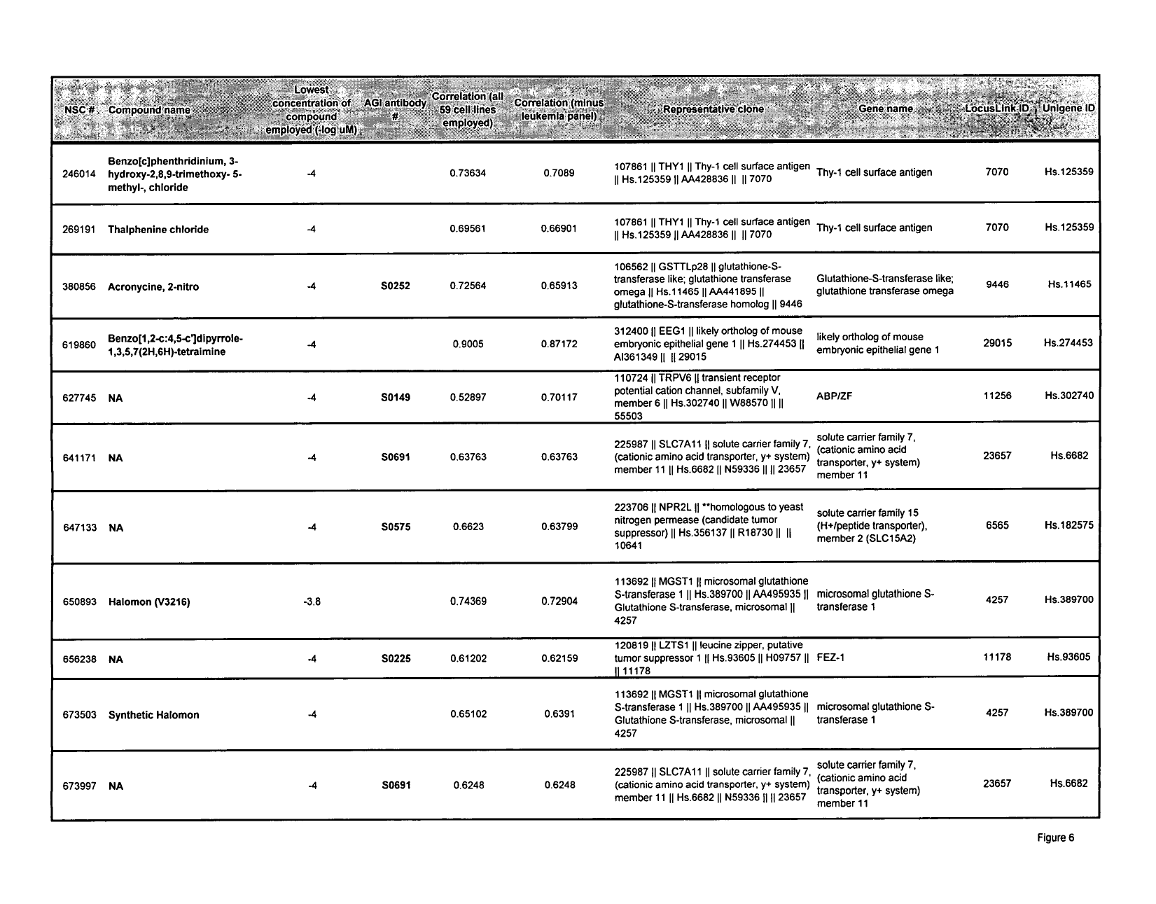

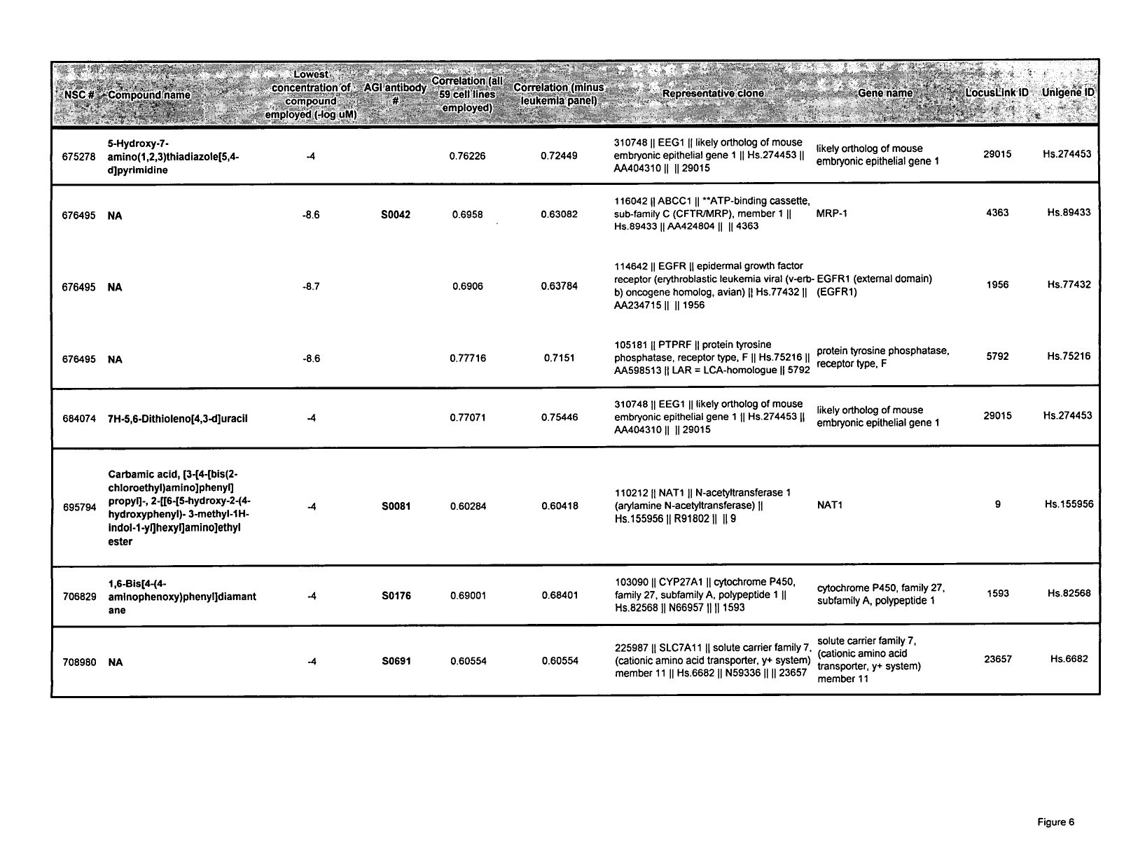

As also set forth in the Examples, the present inventors have defined 30 preferred biomarker:compound pairs that fall into two classes: 1) compounds with very strong correlations with genes expected to be good candidate targets and 2) compounds correlated with genes defined as good targets on the basis of antibody staining data obtained across a large cohorts of tumor samples. FIG. 6 presents these preferred biomarker:compound pairs.

In light of the success demonstrated herein, those of ordinary skill in the art will be able to identify additional biomarker:compound correlates without undue experimentation.

Using Results of Correlative Analysis

The biomarker:compound correlations established according to the present invention may be employed to classify tumor cells as likely or unlikely to respond to particular chemotherapeutic agents, and/or to identify particular chemical compounds that are promising chemotherapeutic agents for use on tumor cells expressing a particular biomarker.

Classifying Tumor Samples as Responsive or Unresponsive to Correlated Compounds

Once a biomarker:compound correlation has been established according to the present invention, it can be relied upon in diagnostic assays to classify tumor samples or cells as likely or unlikely to be growth inhibited by the particular compound (or related compounds), based on detection of the biomarker in the tumor samples or cells.

In general, the source and preparation of the tumor sample or cells is not critical to the present invention. In some cases, however, it may be desirable to prepare and process a patient's tumor sample/cells in a manner as closely analogous to that by which the experimental tumor sample/cells were processed when the correlation was initially established. This strategy may maximize the likelihood that detection of, or failure to detect, the biomarker in the patient's sample reflects the correlation.

Tumor samples or cells for diagnostic analysis in accordance with the present invention may be obtained from any available source, but preferably from a human or animal patient. Preferred animals include domesticated animals and pets such as, for example, cats, dogs, pigs, sheep, cows, horses, etc. Particularly preferred samples are obtained from a human source.

Tumor samples may be processed by any technique known in the art that renders the cells available for detection of a correlated biomarker. The biomarker may then be detected by any available means. In certain preferred embodiments, the biomarker in the patient sample is detected by the same means used to detect the biomarker in establishing the biomarker:compound correlation. In other embodiments, the biomarker is detected by detecting an event or product in biomarker gene expression, such as, for example, transcription, post-transcriptional processing, splicing, nuclear export, translation, post-translational modification, and/or subcellular localization. In other embodiments, the biomarker is detected by assessment of gene copy number (e.g., using fluorescence in situ hybridization (FISH), which in some cases is associated with expression. In yet other embodiments, the biomarker is detected by means of an activity assay (e.g., a binding or enzymatic activity assay).

In certain preferred embodiments of the invention, the biomarker is detected by hybridization to an RNA product of the biomarker gene (or to a DNA copy of the RNA product). In other preferred embodiments, the biomarker is detected by binding of a specific ligand (e.g., an antibody) to the biomarker in the tumor sample. In particularly preferred embodiments, the antibody is a monoclonal or polyclonal antibody. The antibody may be detectably labeled with a marker that releases (directly or indirectly) a signal such as, for example, a fluorescent, chemiluminescent, colorimetric, or radiographic signal.

As discussed above, the present invention provides a variety of biomarker:compound pairs that are either positively (FIG. 4) or negatively (FIG. 5) correlated. In accordance with the present invention, detection of a biomarker of FIG. 4 in a tumor sample classifies the tumor from which the sample originated as likely to respond to treatment with any or all compounds positively correlated with the biomarker. According to the present invention, detection of a biomarker of FIG. 5 in a tumor sample classifies the tumor from which that sample was obtained as unlikely to respond to treatment with any of the negatively correlated compounds.

Identifying Compounds that Inhibit Growth of Cells Expressing Correlated Biomarkers

The techniques, information, and reagents of the present invention may also be employed to identify new or different chemical compounds potentially useful in the treatment of particular tumors.

For example, as discussed above and in the Examples, the present inventors have identified 30 preferred biomarker:chemical compound pairs that fall into two classes: 1) compounds with very strong correlations with genes expected to be good candidate targets and 2) compounds correlated with genes defined as good targets on the basis of antibody staining data obtained across a large cohorts of tumor samples. It will be appreciated that, as described in the Examples, the filters applied to the biomarker expression and/or compound toxicity data points were slightly different when the goal was to identify new candidate therapeutic agents rather than diagnostic indicators.

For example, data points for compounds that showed dramatic activity differences across cell lines were favored. Also, data points for compounds that showed very potent toxicity for one or more cell lines were favored. Other factors that were considered included, for example, whether the biomarker was a cell surface protein and therefore more likely to be available for interaction with a chemical compound in vivo. Each of these factors was intended to bias the outcomes in favor of identifying genuine therapeutic candidates. Additional factors that could be considered include, for example, structure or activity features of the compound expected to affect bioavailability or stability, solubility or characteristics of the compound expected to affect formulation of the compound, features of the compound which are expected to affect absorption, distribution, metabolism, and/or excretion of the compound could also be considered, as well as other pharmacokinetic considerations, etc.

Thus, according to the present invention, detection of a correlated biomarker in a tumor sample designates that tumor as likely to respond to the particular compound. Conversely, the correlation designates the compound as particularly effective for treatment of the tumor. In certain cases, additional preclinical and/or clinical analyses of the compound will be required before it can be administered to humans. Moreover, in many cases, the correlated compound does not represent a clinical agent itself, but rather is a lead compound whose activity is established as being of interest by virtue of the inventive correlation. Those of ordinary skill in the art are well familiar with the process and techniques associated with defining and preparing derivatives and/or analogs of such compounds to be tested as clinical agents.

In some embodiments of the invention, it will be desirable to treat the individual suffering from the tumor only with the correlated compound (or analog or derivative thereof). In other embodiments, it will be desirable to combine treatment with the compound with one or more other treatments, including for example surgical or radiological treatments.

It should be noted also that the present invention provides not only identification of potential new chemotherapeutic agents, but also characterization of such agents, both new and old. For instance, the “therapeutic index” of a pharmaceutical agent is a measure of the range of concentrations at which treatment with the compound will produce a beneficial result without undue toxicities. The present invention defines tumors that are more sensitive to particular compounds, which inherently defines situations in which a lower concentration of compound has stronger effects. The present invention therefore provides information relevant to the assessment of the therapeutic index for cytotoxic compounds.

Identifying Biological Pathways that Confer Sensitivity or Resistance to Chemical Compounds

As indicated herein, many of the biomarker:compound correlations identified in accordance with the present invention will represent direct physical interactions between the biomarker and compound. In other cases, the biomarker will participate in a biological pathway affected by the compound, but will not interact directly with the compound. Inventive identification of multiple biomarkers with a single compound could reveal a common biological pathway for the multiple biomarkers. Comparably, correlation of a compound with known activity (e.g., microtubule binding) with a particular biomarker will, in some cases, reveal the biological pathway in which the biomarker participates.

Kits

The present invention provides kits and sets of reagents for detecting correlated biomarkers in tumor samples. For example, inventive kits may include one or more antibodies to biomarkers correlated with sensitivity (or insensitivity) to particular compound(s). Alternatively or additionally, the kits could include one or more reagents useful for preparing the sample for antibody binding, and/or for detecting such binding.

The invention also provides kits containing reagents for isolating mRNA from a tumor sample, in order to allow gene analysis, for example on a microarray.

The invention further provides kits containing reagents for collecting biomarker expression and/or compound toxicity data points for use in inventive datasets. These kits may optionally include one or more computer programs, for example for filtering or analyzing data.

Pharmaceutical Compositions

As discussed above, the present invention provides chemical compounds that are useful as pharmaceutical agents in the treatment of cancer. In some preferred embodiments of the invention, these chemical compounds are small molecules. In other preferred embodiments, the compounds are antibodies or other specific ligands.

Inventive chemical compounds for use as pharmaceutical agents may be combined with one or more other components and prepared in a formulation for delivery by any route including, for example, orally, rectally, parenterally, intracisternally, intravaginally, intraperitoneally, topically (as by powders, ointments, or drops), bucally, as an oral or nasal spray, or the like, depending on the severity of the infection being treated, to a subject. In most cases, it is expected that inventive compounds will be formulated for oral or parenteral (e.g., i.v.) administration.

Thus, the present invention provides pharmaceutical compositions comprising an inventive chemical compound and a pharmaceutically acceptable carrier. The composition may optionally comprise one or more additional therapeutic agents (e.g., pain relievers, other chemotherapeutic agents, etc.).

It will be appreciated that inventive compounds may be incorporated into the pharmaceutical compositions in an alternate form such as for example, as a pharmaceutically acceptable salt, or as a prodrug. For example, pharmaceutically acceptable derivatives of inventive compounds generally include pharmaceutically acceptable salts, esters, salts of such esters, or any other adduct or derivative that, when administered to a patient, in provides to that patient, directly or indirectly, a compound as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19, 1977, incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; Cremophor; Solutol; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of the present invention generally contain a therapeutically effective amount of the inventive compound in light of the other components of the composition, or other medications the patient may be receiving. In certain embodiments of the present invention a “therapeutically effective amount” of the inventive compound or pharmaceutical composition is that amount effective for killing or inhibiting the growth of tumor cells. For example, the amount effective to kill 50%, 90%, 95%, or 99% of the cells in a cell culture such as described below in the Examples. As is known in the art, the exact amount required will vary from subject to subject, depending on the species, age, and general condition of the subject, the severity of the infection, the particular anticancer agent, its mode of administration, and the like.

The anticancer compounds of the invention are preferably formulated in dosage unit form for ease of administration and uniformity of dosage. The expression “dosage unit form” as used herein refers to a physically discrete unit of anticancer agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, the compounds of the invention may be administered orally or parenterally at dosage levels of about 0.01 mg/kg to about 100 mg/kg, preferably from about 0.1 mg/kg to about 50 mg/kg, and more preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the liquid compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of an inventive compound, it is often desirable to slow absorption after subcutaneous or intramuscular injection. This may be accomplished by the use of a liquid suspension of crystalline or amorphous material with poor water solubility. The rate of absorption of the compound then depends upon its rate of dissolution which, in turn, may depend upon crystal size and crystalline form. Alternatively, delayed absorption of a parenterally administered compound is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of release can be controlled. Examples of other biodegradable polymers include poly(orthoesters) and poly(anhydrides). Depot injectable formulations are also prepared by entrapping the compound in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active compound is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As noted herein, it will be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with the inventive compounds of the present invention include surgery, radiotherapy (in but a few examples, radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. A more comprehensive discussion of updated cancer therapies can be found at www.nci.nih.gov. A list of the FDA approved oncology drugs can be found at www.fda.gov/cder/cancer/druglistframe.htm and in The Merck Manual, Seventeenth Ed. 1999, the entire contents of which are hereby incorporated by reference.

In another aspect, the present invention provides combination therapies comprising compound of the invention and medication known to combat the side effects of these compounds. For example, medication which relieves pain, anemia, nausea, hair loss, lethargy, etc. may be combined with the inventive compounds in a therapeutic combination. In particular, pain or nausea medication may be combined with the inventive compounds.

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

### EXAMPLES

### Example 1

### Establishing Gene Expression and Drug Sensitivity Patterns Across Tumor Cell Lines

The present Example describes the inventors' establishment of gene expression pattern profiles containing information on approximately 17,000 unique genes for 59 different tumor cell lines, and also presents compound sensitivity data for approximately 40,000 compounds tested by the National Cancer Institute against the same cell lines.

Materials and Methods

PRODUCTION OF CDNA MICROARRAYS. The 44,000 human cDNA clones used in these experiments were obtained from Research Genetics (Huntsville, Ala.) as bacterial colonies in 96-well microtiter plates. Each insert was amplified from a bacterial colony by sampling one microliter of bacterial media and performing polymerase chain reaction amplification of the insert using consensus primers for the three plasmids represented in the clone set (5′-TTGTAAAACGACGGCCAGTG-3′, SEQ ID NO:1 and 5′-CACACAGGAAACAGCTATG-3′, SEQ ID NO:2). Each 100 μl PCR product was purified by precipitation, and resuspended in 3×SSC. The PCR products were then printed on treated poly-lysine coated glass microscope slides using a robot with thirty-two printing tips. Detailed protocols for assembling and running a microarray spotter are publicly available online at cmgm.stanford.edu/pbrown.

PREPARATION OF MRNA AND REFERENCE POOL. Cell lines were grown from NCI Developmental Therapeutics Program frozen stocks in RPMI-1640 supplemented with phenol red, 2 mM glutamine, and 5% fetal calf serum. The time between removal from the incubator and lysis of the cells in RNA stabilization buffer was minimized (less than one minute). Cells were lysed and RNA purified by use of the Invitrogen Inc. FASTRACK kit according to manufacturer's instructions. mRNA from the following cells was combined in equal quantities to make the reference pool: MCF7 (Breast carcinoma), Hs578T (Breast carcinosarcoma), NTERA2 (Teratoma), colo 205 (colon carcinoma), ovcar3 (ovarian carcinoma), UACC-62 (melanoma), Molt-4 (T-cell leukemia), RPMI-8226 (Multiple Myeloma), NB4 treated for 24 hours with 1 μM All-trans retinoic acid (myeloid differentiated leukemia), SW-872 (liposarcoma) and HepG2 (Liver carcinoma).

PREPARATION AND HYBRIDIZATION OF FLUORESCENT LABELED CDNA. For each comparative array hybridization, labeled cDNA was synthesized by reverse transcription from test cell mRNA in the presence of Cy5-dUTP, and from the reference mRNA with Cy3-dUTP, using the Superscript II Reverse-transcription kit. For each reverse transcription reaction, 2 μg of mRNA was mixed with 2 μg of an anchored oligo-dT (d-20T-d(AGC)) primer in a total volume of 15 μl, heated to 70° C. for 10 minutes and cooled on ice. To this sample were added 0.6 μl of an unlabeled nucleotide pool (25 mM each dATP, dCTP, dGTP, and 15 mM dTTP), 3 μl of either Cy3 or Cy5 conjugated dUTP (1 mM) (Amersham), 6 μl of 5× first-strand buffer (250 mM Tris-HCL (pH8.3), 375 mM KCl, 15 mM MgCl₂), 3 μl of 0.1M DTT, and 2 μl of Superscript II reverse transcriptase (200 μg/μl) (Gibco-BRL). After a two-hour incubation at 42° C., the RNA was degraded by addition 1.5 μl of 1N NaOH, and incubation at 70° C. for 10 minutes. The mixture was neutralized by addition of 1.5 μl of 1N HCL, and the volume brought to 500 μl with TE (10 mM Tris, 1 mM EDTA). 20 μg of Cot1 human DNA (Gibco-BRL) was added, and the probe was purified by centrifugation in a Centricon-30 micro-concentrator (Amicon). The two separate probes were combined, brought to volume of 500 μl, and concentrated again to a volume of less than 7 μl. 2 μL of 10 μg/μl polyA RNA (Sigma) and 20 μg/ul tRNA (Ambion) were added, and the volume adjusted to 35 μl with distilled water. For final probe preparation, 2.1 μl 20×SSC (1.5M NaCl, 150 mM NaCitrate (pH8.0)) and 0.35 μl 10% SDS were added to a total final volume of 12 μl. The probes were denatured by heating for 2 minutes at 100° C., incubated at 37° C. for 20-30 minutes, and placed on the array under a 22 mm×22 mm glass cover slip. The slides were incubated overnight at 65° C. for 14 to 18 hours in a custom slide chamber with humidity maintained by a small reservoir of 3×SSC. Arrays were washed by submersion and agitation for 2-5 minutes in 2×SSC with 0.1% SDS, followed by 1×SSC, and then 0.1×SSC. The arrays were “spun dry” by centrifugation for 2 minutes in a slide rack in a Beckman GS-6 tabletop centrifuge in Microplus carriers at 650 RPM for 2 minutes.

ARRAY QUANTITATION AND DATA PROCESSING. Following hybridization, arrays were scanned using a laser-scanning microscope. Separate images were acquired for Cy3 and Cy5. For each fluorescent image, the average pixel intensity within each circle was determined, and a local background was computed for each spot equal to the median pixel intensity in a radius of 20 pixels around the spot center, excluding all pixels within other defined spots. Net signal was determined by subtraction of this local background from the average intensity for each spot. Spots deemed unsuitable for accurate quantitation because of array artifacts were manually flagged and excluded from further analysis. Data files were entered into a custom database that maintains web accessible files. Signal intensities between the two fluorescent images were normalized by applying a uniform scale factor to all intensities measured for the Cy5 channel. The normalization factor was chosen so that the mean log(Cy3/Cy5) for a subset of spots that achieved a minimum quality parameter (approximately 6000 spots) was 0. This effectively defined the signal-intensity-weighted “average” spot on each array to have a Cy3/Cy5 ratio of 1.0.

Results

The present inventors measured the expression of 17,000 genes across 59 of the 60 standard tumor derived cell lines assembled by the National Cancer Institute (the NCI60) as models for tumors of diverse tissue origins. The cell lines included in this panel are listed in Appendix A (see also dtp.nci.nih.gov/docs/misc/common_files/cell_list.html).

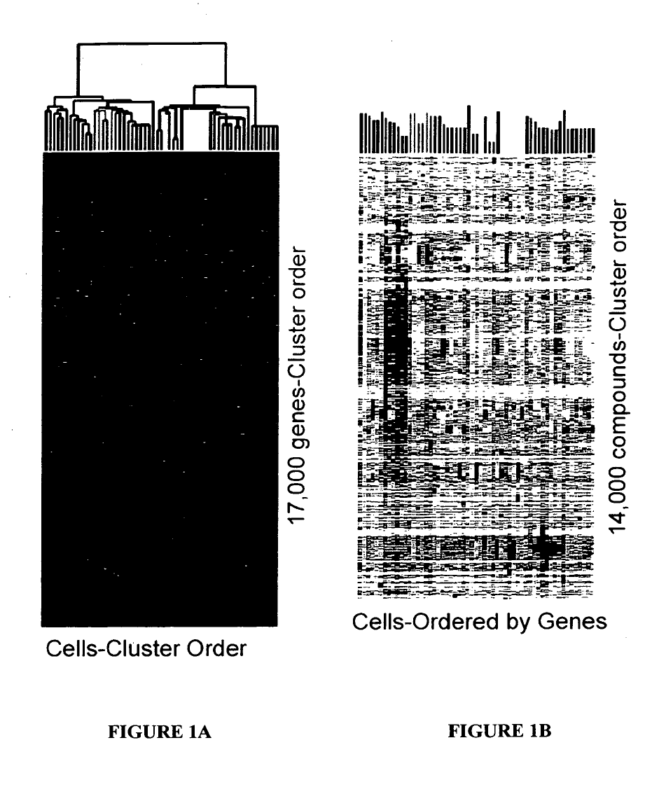

The present inventors established a gene expression profile for each of the 59 publicly-available NCI60 cell lines. Specifically, the inventors grew each of the NCI60 cell lines in a controlled tissue culture environment, and isolated mRNA from exponentially growing cells. They generated a “common reference” from eleven of the cell lines, and also generated fluorescently labeled cDNA from each of the 59 mRNA samples. The common reference cDNA was co-hybridized with an experimental cDNA sample from a single cell line to a spotted cDNA microarray containing more than 44,000 ESTs to measure gene expression variation in each of the 59 cell lines. The data were filtered to generate a high quality dataset. Specifically, cDNAs that were detectable in fewer than 80% of the cell lines were discarded; approximately 17,000 unique genes were left whose measurements were recorded. FIG. 1A shows the patterns of variation of these 17,000 genes across the cell lines revealed by hierarchical cluster analysis of the gene expression database. As expected, the cell lines cluster into groups that reflect their tissue of origin.

The Developmental Therapeutic Program (DTP) at NCI has used the NCI60 panel of cell lines to identify cytotoxic agents that show some tissue specificity (see, for example, Monks, et. al. J. Natl Cancer Inst 83(11):757-66, 1991; Paull, et. al. J. Natl Cancer Inst 81(14):1088-92, 1989, each of which is incorporated herein by reference). The DTP has now tested more than 1 million compounds for toxicity to the NCI60 cell lines, and has made public their results for over 40,000 compounds that showed some interesting variation in activity across the panel. Datasets that contain the GI50 (i.e., the concentration of a compound that inhibits growth of a particular cell line by 50% after 48 hours of exposure) for particular compounds linked to compound identification information can be downloaded from the DTP website (dtp.nci.nih.gov/dtpstandard/cancerscreeningdata/indexjsp). The datasets include information for most common therapeutic agents (termed “Standard Agents” by the NCI), as well as several natural product extracts.

As shown in FIG. 1B, the present inventors have performed hierarchical cluster analysis of the GI50 compound sensitivity database. The cell lines are ordered by clustering across the gene expression data in both cluster diagrams. A simple visual comparison of FIG. 1B with the gene expression patterns seen in FIG. 1A suggests that aspects of the cell lines′ physiology are reflected in the patterns of variation in the two datasets. For example, a large number of genes are highly expressed in leukemia cell lines (columns designated by red bars), and a large number of compounds are particularly toxic to leukemia cell lines. Thus, expression of the leukemia-specific genes correlates with toxicity of the compounds to leukemia cell lines.

Without wishing to be bound by any particular theory, the present inventors propose that many of the genes whose expression correlates with responsiveness to a particular compound represent markers of biologic pathways that are involved in responding to the compound. It is likely that a subset of the gene expression:compound correlations result from direct interaction between the compound and the gene product. For this subset of correlations, compounds can be identified whose cytotoxic effects are dependent upon the expression of a single gene. For other gene expression:compound correlations, the genes are candidate biomarkers whose expression correlates with compound sensitivity even though there may not be any direct physical interaction between the compound and any gene product. According to the present invention, when these candidate biomarkers are similarly regulated across tumor samples, they are useful correlated biomarkers for predicting responsiveness of a given tumor cell or sample to their correlated pharmaceutical compound.

### Example 2

### Detecting a Correlation Between a Known Gene and an Interacting Compound

The present Example establishes the ability of the inventive strategy to establish correlations between genes whose expression varies across tumor cell lines and compounds whose toxicity also varies across tumor cell lines, and whose toxocity is due at least in part to an interaction between the compound and a product of the relevant gene.

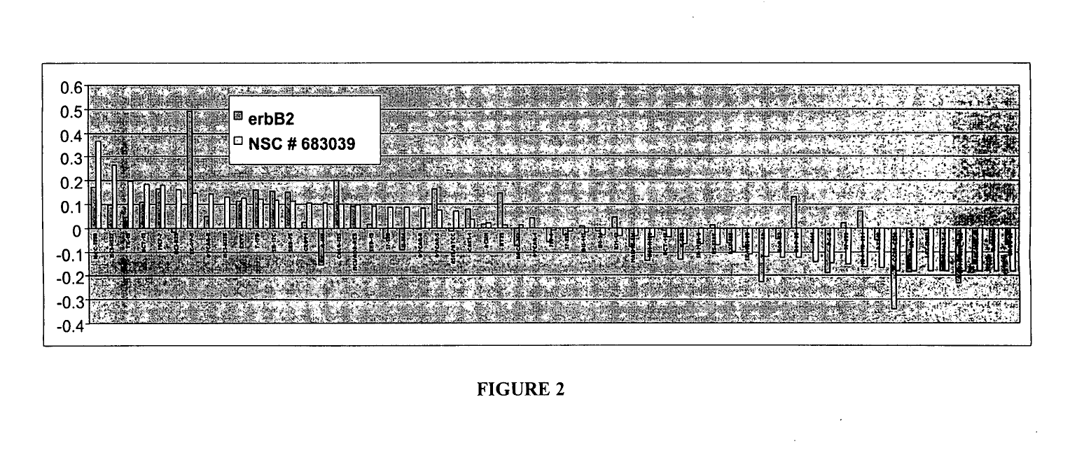

The inventors used normalized gene expression data for the erbB2 gene across the 59 cell lines to identify from a dataset of 14,542 compounds those that had the most highly correlated compound toxicity pattern. The pattern of erbB2 expression was highly correlated with a number of different compounds. The compound showing the strongest correlation, compound number 683039, is a conjugate of an anti-erbB2 antibody (specifically an anti-c-erbB2 disulfide-stabilized Fv fragment) fused to a cytotoxin (specifically a truncated form of Pseudomonas exotoxin). A comparison of the normalized pattern of erbB2 gene expression with the normalized GI50 values for compound 683039 is shown in FIG. 2. The Pearson correlation coefficient between the erbB2 gene expression pattern and the pattern of variation of the GI50 for 683039 is 0.61. This demonstration that the inventive methods identify, out of a pool of more than 14,000 compounds, an antibody-toxin conjugate specific for erbB2 as the chemical agent whose cytotoxic activity most strongly correlates with erbB2 expression provides compelling proof of principle that the inventive biomarker:compound correlates have strong diagnostic and therapeutic relevance.

### Example 3

### Detecting a Correlation Between a Known Compound with Known Activity and a Gene Responsible for Resistance to the Compound

This Example demonstrates, among other things, that the present invention can identify negative correlations between cytotoxic compounds and genes whose products impart resistance to the compounds.

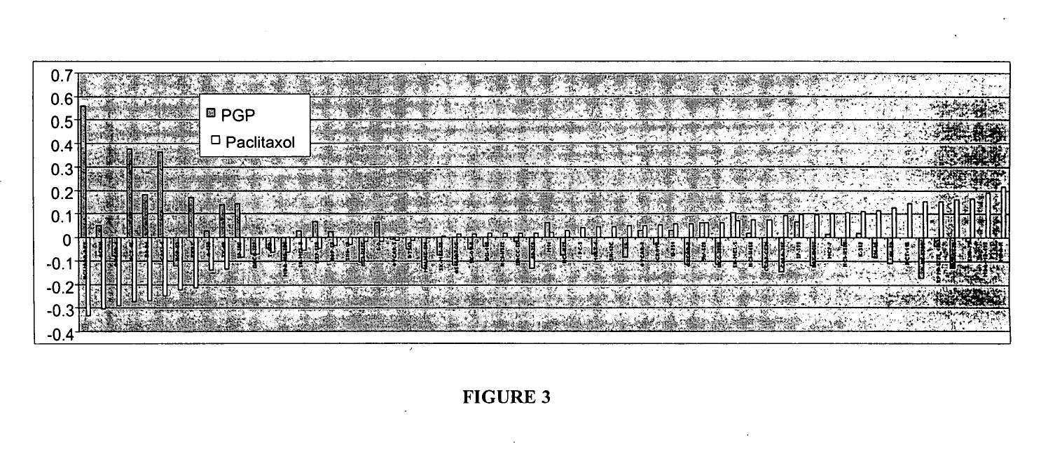

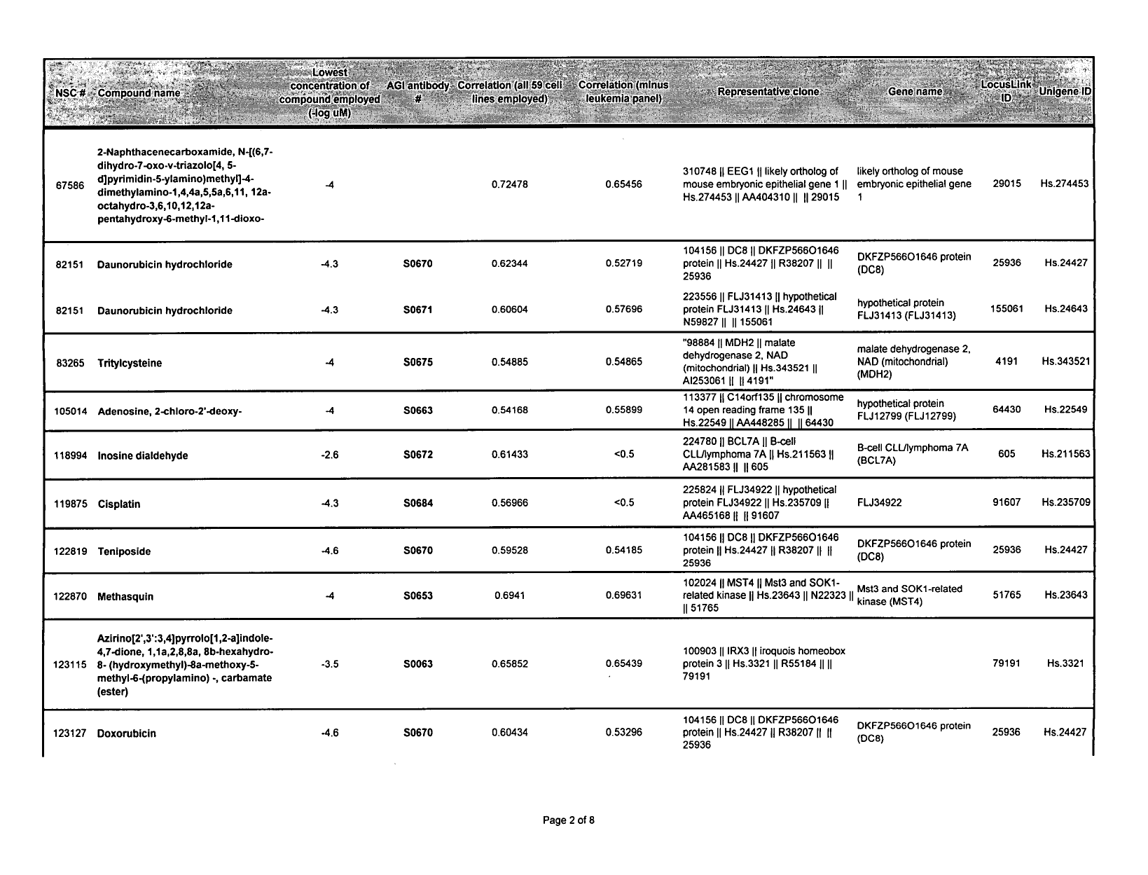

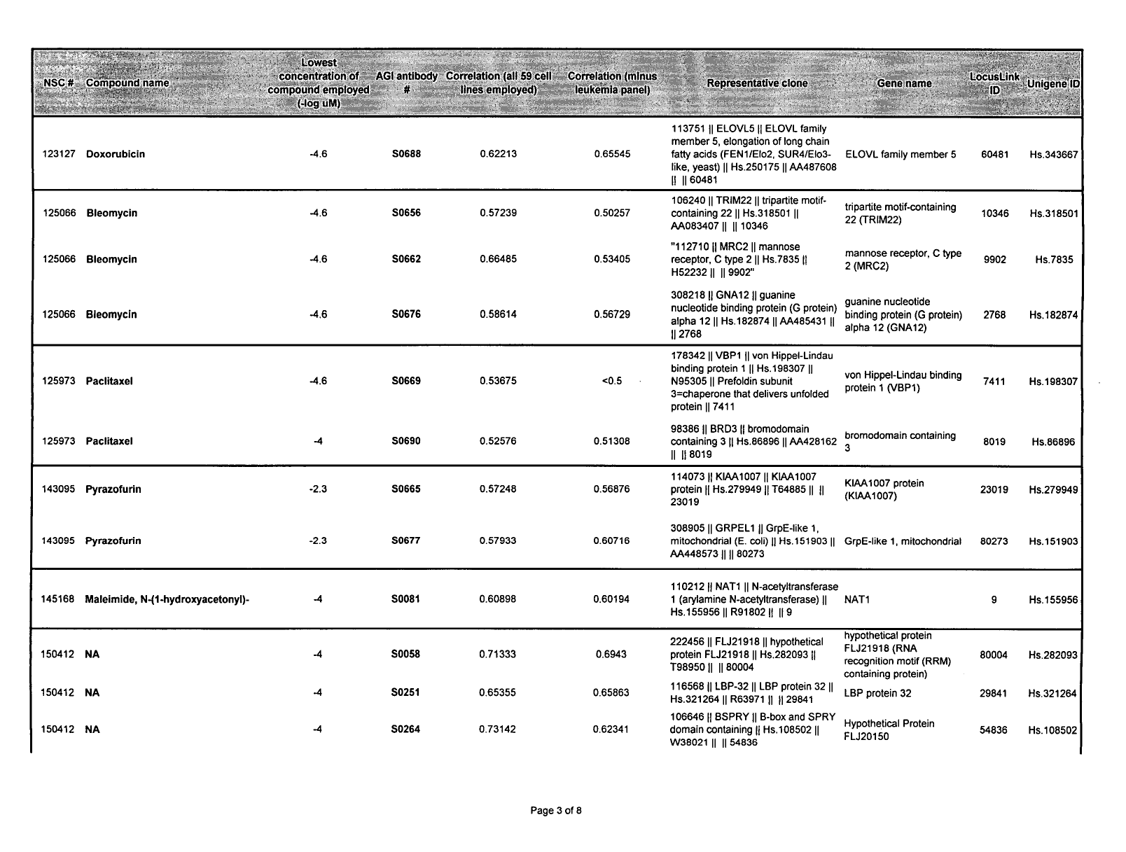

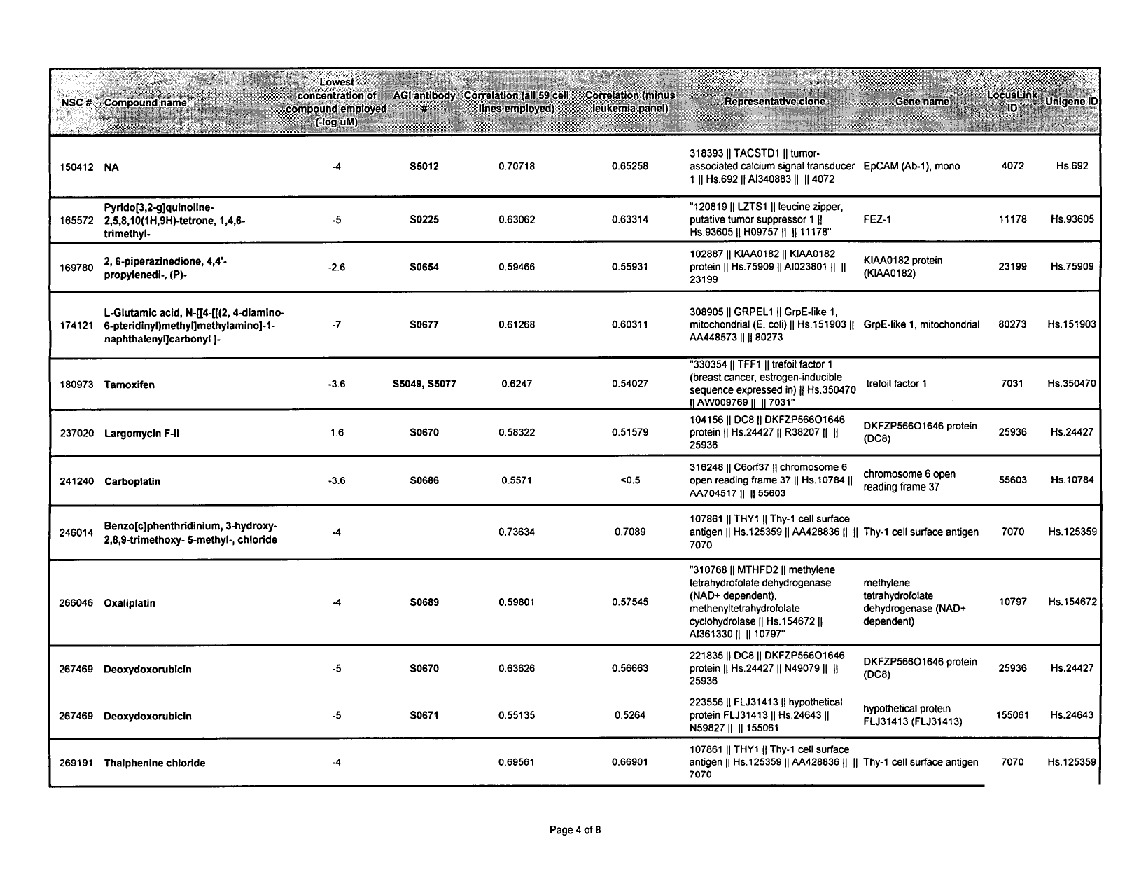

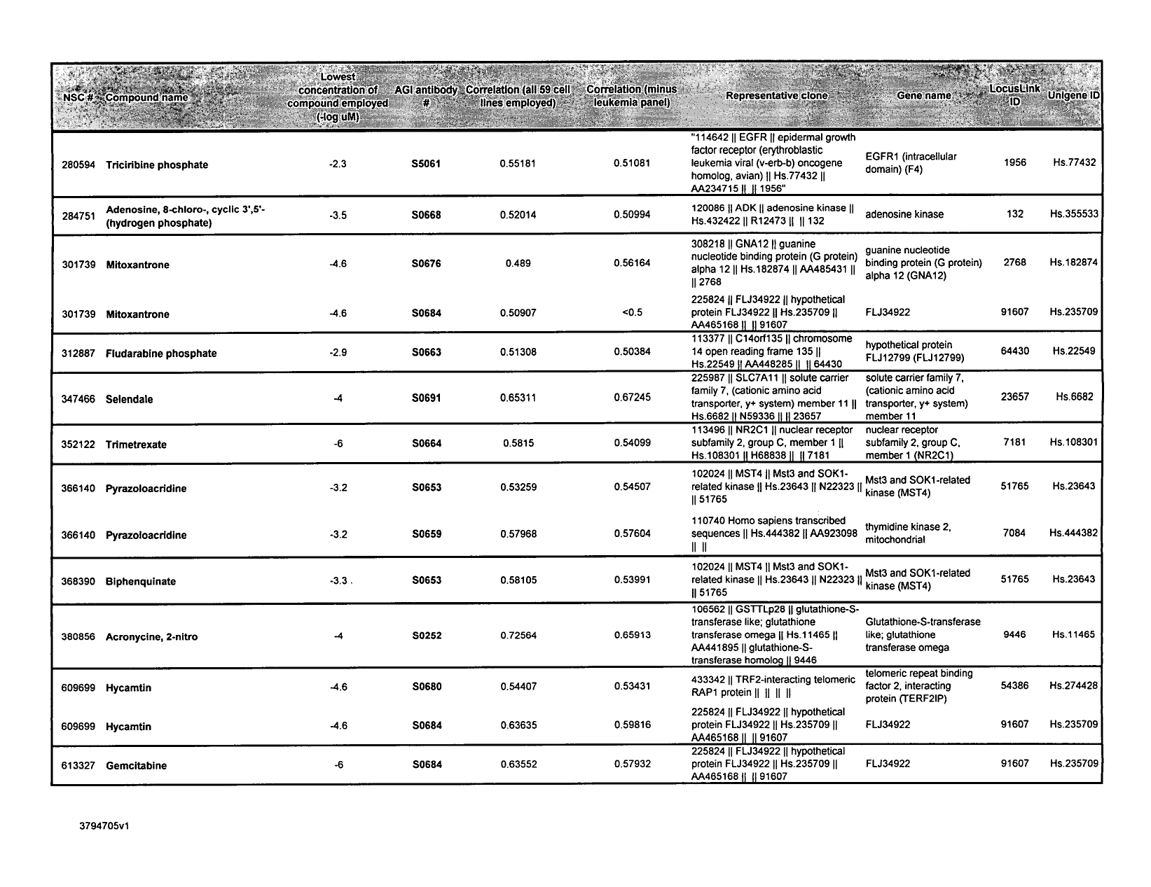

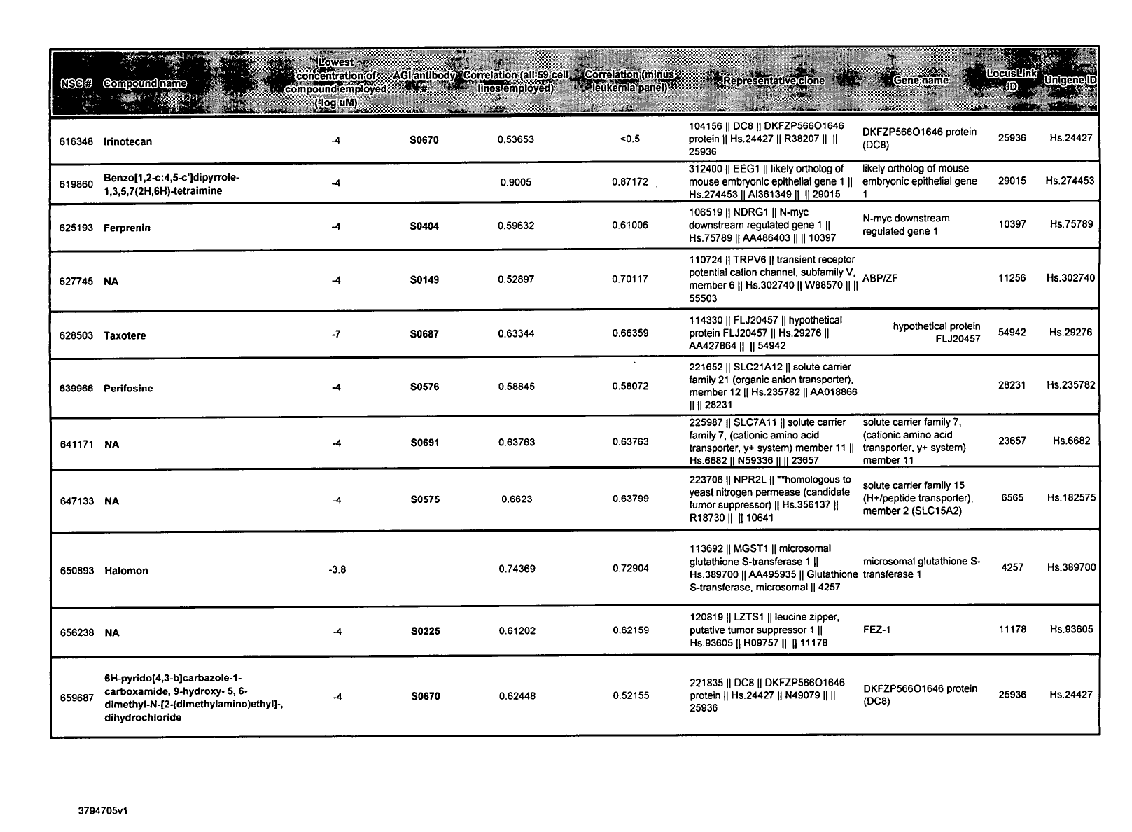

The present inventors used the pattern of toxicity displayed by Paclitaxol to the NCI60 cell lines to query the gene expression database and identify genes with the most strongly anti-correlated expression patterns. As shown in FIG. 3, the gene with the highest negative correlation was ABCB1, the multidrug resistance p-glycoprotein pump (“PGP”) that is known to impart resistance to Paclitaxol. Furthermore, consistent with the role that PGP plays in removing toxic compounds from cells, the inventors found that PGP expression is significantly negatively correlated with the toxicity patterns of 130 different compounds. An average gene would be expected to shown a negative correlation with only 4 compounds. These results demonstrate that, for at least some biomarker:compound pairs, inventive gene expression:compound toxicity correlations are strong measures of target-directed activity.

### Example 4

### Filtering Datasets

The present Example describes the effects of various processing steps imposed on initial datasets, so that manageable numbers of reliable correlations were achieved.

In order to generate a “high quality” compound toxicity pattern dataset to be compared with the inventive “high quality” filtered gene expression dataset described in Example 1, the present inventors excluded from the dataset provided by the DTP all information for compounds for which measurements had been obtained in fewer than 80% of the cell lines. Information for compounds that showed little or no variation in toxicity across the cell was also excluded by restricting those compounds for which the spread of the data (the maximum GI50 value minus the minimum GI50 value) was less than the average variation found within repeat measurements of the cytotoxicity within each cell line for that compound. The resulting “high quality compound toxicity dataset” contained information for approximately 21,000 compounds.

The Pearson correlation was determined for all cDNAs in the “high quality” 20 filtered gene expression dataset and compounds in the “high quality” compound toxicity dataset (357,000,000 measurements). Approximately 1.5 million correlates with correlations of greater than 0.5 were identified; preferred correlations were stronger than 0.6. A strong component of the data generated by this analysis reflected genes and compounds that are differentially expressed or differentially active in leukemia cell lines as contrasted with other cell lines. This effect may well reflect the broad physiological differences between leukemia cell lines, which are grown in suspension or under adherent conditions, as contrasted with other cell lines. The detected correlations between these genes and compounds may have little relevance to the biological mechanism of action of the compound and its relationship to the biological role of the gene product. The correlation analysis was therefore also performed without the data from the leukemia cell lines.

Preferred correlate pairs for immediate analysis were selected by application of certain inventive principles. For instance, correlate pairs involving compounds with more potent toxicity were favored when analyzing the data, with the goal of identifying potential novel lead compounds for therapeutic development. In these cases, a minimal potency of the compound (as measured by the GI50 assessment of cytotoxicity) was preferred (e.g., a maximum GI50 of at least 10⁻⁶ M). Compounds whose ability to achieve GI50 varied minimally around a large concentration were ignored.

Additionally, correlations established on the basis of data from a large number of cell lines were favored. Kurtosis, which measures the size of the distribution of data within the dataset, was invoked to quantify this preference. A kurtosis value of greater than 15 was often used as a filter for compounds which had few cell lines contributing to the variability.

Furthermore, efforts were made to exclude correlations that, like the leukemia bundle mentioned above, were likely to reflect broad physiological differences between or among tumor cell types rather than specific interactions between gene products and compounds. A variety of gene expression data sources (e.g., Ross et al., Nat Genet. 24:227, 2000) were examined to identify such large scale gene expression phenomena (e.g., genes expressed during proliferation).

As discussed above in Examples 2 and 3, the inventive process identified expected biomarker:compound correlate pairs such as erbB2 exrpression:anti-erbB2 conjugate sensitivity (Example 2) and PGP expression:Paclitaxel insensitivity (Example 3). Other expected correlations (e.g., EGFR:TP4EK sensitivity, see, for example, Wosikowski et al., J. Natl Cancer Inst. 89:1505-1515,1997) were also identified. Overall, the process through which the correlations (positive and negative) were established and prioritized may be outlined as follows:
1) about 1.6 billion correlated pairs based upon raw datasets.
2) about 300 million pairs based upon filtered datasets.
3) about 1.5 million pairs stronger than about 0.5.
4) about 1.1 million pairs resulting from data for which >75-80% of the cell lines have both gene expression and cytotoxicity measurements.
5) about 1 million pairs having kurtosis values<15.
6) about 75,000 pairs for which the maximum and minimum GI50 values vary by at least two logs.
7) about 15,000 pairs for which the correlation is stronger than 0.5 in the absence of the leukemia cell line data.
8) About 3,000 for which the correlation is stronger than 0.6.

### Example 5

### Identifying Biomarkers Correlated with Sensitivity or Insensitivity to Toxins

This Example describes the inventive identification of 111 biomarker:compound pairs that reflect and/or predict sensitivity or insensitivity of tumor cells expressing the biomarker to the correlated compound. These pairs include 77 different compounds and 69 distinct genes.

The compound sensitivity dataset provided by the DTP was expanded with the addition of publicly available information about additional compounds in clinical use or development (additional compounds are listed in Appendix D), and correlations were detected with the gene expression dataset across the NCI60 panel. As discussed above in Example 4, kurtosis was used to identify correlates supported by data from a large number of cell lines. Specifically, compounds for which the kurtosis value was above 15 were excluded (those of ordinary skill in the art will appreciate that genes for which kurtosis values were too high could also have been excluded). This analysis produced about 2000 biomarker:compound correlate pairs. These pairs were ranked according to the average of the correlation with and without the leukemia panel. The pairs were further examined to exclude those for which the correlation was likely due to some general physiologic event (e.g., cell proliferation). Also correlations for which the underlying data showed significant scatter were excluded. Finally, poorly expressed genes (determined by analysis of the average magnitude of expression of the gene via consideration of the signal channel intensity in the cDNA array hybridization) were discarded.

This analysis identified both genes whose expression positively correlates with compound sensitivity and genes whose expression negatively correlates with compound sensitivity. FIG. 4 is a Table of positively correlated genes and compounds (91 pairs consisting of 71 compounds and 55 genes); FIG. 5 is a Table of negatively correlated genes and compounds (20 pairs consisting of 15 compounds and 15 genes). According to the present invention, genes that are positively correlated may be useful in the identification of patients who are likely to respond to treatment with the relevant compound; conversely, genes that are negatively correlated may be useful in the identification of patients who are unlikely to respond to treatment with the relevant compound.

The present inventors have raised or obtained antibodies to proteins encoded by several of the genes (indicated in the AGI antibody column) listed in FIGS. 4 and 5. According to the present invention, such antibodies can be included in diagnostic kits for the analysis of tumor samples from patients.

### Example 6

### Identification of Chemical Compounds Useful in Chemotherapy of Tumor Cells Expressing Correlated Biomarkers

The present Example describes the inventive identification of candidate therapeutic agents for the treatment of tumors expressing certain genes.

From the 1.5 million biomarker:compound correlates identified in Example 4, the present inventors selected a set of 30 correlates that fell into two classes: 1) compounds with very strong correlations with genes expected to be good candidate targets and 2) compounds correlated with genes defined as good targets on the basis of antibody staining data obtained across a large cohorts of tumor samples. The mathematical criteria and selection process for these biomarker:compound correlates was as described above, except that only positive correlations were considered. In general, the magnitude of the correlation was higher than that seen above in Example 5. Specifically, the average correlation was greater than 0.68. Additionally, the magnitude of the difference between sensitive and resistant cell lines was considered when ranking the desirability/interest of the inventive methods and reagent in order to ensure that the compounds chosen exhibited markedly different toxicities across the cell lines and showed potencies more likely to be able to serve as lead compounds with sufficient toxicity in the targeted tumor. Thus, the highest acceptable kurtosis for the GI50 measurement was lowered to 5 for these correlate pairs in order to select for the “quality” of the correlative relationship. This analysis generated a list of 30 correlates consisting of 25 compounds and 17 genes (in some cases, multiple compounds were directed at the same gene(s)). These preferred biomarker:compound pairs are listed in FIG. 6.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

### Appendix D

Compounds used to establish the compound dataset of Example 5
5FU
Alemtuzumab (Campath)
Bevacizumab (Avastin)
bleomycin
Carboplatin
cisplatin
Cyclophosphamide
dactinomycin
daunorubicin
DHAD (Mitoxantrone)
doxorubicin
Epirubicin
Erbitux (cetuximab)
etoposide
Gemcitabine (Gemzar)
Gleevec
Herceptin
Ifosfamide
Irinotecan
Melphalan
methotrexate
Mitomycin
Oxaliplatin
Paclitaxel (Taxol)
Rituximab (Rituxan)
Tamoxifen
Taxotere (Docetaxel)
Teniposide
Topotecan (Hycamtin)
Toremifene citrate
Vinblastine sulfate
Vincristine sulfate

ZD 1839 (Iressa)

APPENDIX C
Developmental Therapeutics Program NCI/NIH
dtp.nci.nih.gov/docs/cancer/searches/standard_agent_table.html
DTP Human Tumor Cell Line Screen
The Standard Agents

| Compound | NSC Number |
|---|---|
| methotrexate | 740 |
| busulfan | 750 |
| thioguanine | 752 |
| 6-mercaptopurine | 755 |
| nitrogen mustard | 762 |
| guanazole | 1895 |
| R-methylformamide | 3051 |
| actinomycin D | 3053 |
| chlorambucil | 3088 |
| thiadiazole | 4728 |
| thio-tepa | 6396 |
| DON | 7365 |
| melphalan | 8806 |
| triethylenemelamine | 9706 |
| hexamethylenemelanime | 13875 |
| gallium nitrate | 15200 |
| 5-fluorouracil | 19893 |
| thymidine | 21548 |
| delta-1-testololactone | 23759 |
| mitramycin | 24559 |
| pipobroman | 25154 |
| cyclophosphamide | 26271 |
| mitomycin C | 26980 |
| 5-FUDR | 27640 |
| hydroxyurea | 32065 |
| methyl-GAG | 32946 |
| uracil nitrogen mustard | 34462 |
| O6-methylguanine | 37364 |
| o,p′-DDD | 38721 |
| DTIC | 45388 |
| vinblastine sulfate | 49842 |
| IMPY | 51143 |
| porfiromycin | 56410 |
| chromomycin A3 | 58514 |
| cytosine arabinoside | 63878 |
| vincristine sulfate | 67574 |
| thalicarpine | 68075 |
| B-TGDR | 71261 |
| A-TGDR | 71851 |
| fluorodopan | 73754 |
| D-tetrandrine | 77037 |
| procarbazine | 77213 |
| CCNU | 79037 |
| daunorubicin (daunomycin) | 82151 |
| S-trityl-L-cysteine | 83265 |
| streptozoticin | 85998 |
| methyl-CCNU | 95441 |
| PCNU | 95466 |
| hexamethylenebisacetamide | 95580 |
| 3HP | 95678 |
| Yoshi-864 | 102627 |
| 5-azacytidine | 102816 |
| cytembena | 104801 |
| 5HP | 107392 |
| L-asparaginase | 109229 |
| iphosphamide | 109724 |
| pentamethylmelamine | 118742 |
| diglycoaldehyde | 118994 |
| cisplatin | 119875 |
| VM-26 (teniposide) | 122819 |
| doxorubicin (Adriamycin) | 123127 |
| bleomycin | 125066 |
| paclitaxel (Taxol) | 125973 |
| dichloroallyl lawsone | 126771 |
| 3-deazauridine | 126849 |
| 5-azadeoxycytidine | 127716 |
| triazinate | 127755 |
| ICRF-159 | 129943 |
| dianhydrogalatitol | 132313 |
| indicine N-oxide | 132319 |
| rifamycin SV | 133100 |
| piperazinedione | 135758 |
| soluble Baker's Antifol | 139105 |
| emofolin sodium | 139490 |
| anguidine | 141537 |
| VP-16 (etoposide) | 141540 |
| homoharringtonine | 141633 |
| hycanthone | 142982 |
| pyrazofurin | 143095 |
| cyclocytidine | 145668 |
| ftorafur | 148958 |
| hydrazine sulfate | 150014 |
| L-alanosine | 153353 |
| maytansine | 153858 |
| neocarzinostatin | 157365 |
| AT-125 (acivicin) | 163501 |
| rubidazone | 164011 |
| bruceantin | 165563 |
| asaley | 167780 |
| ICRF-187 | 169780 |
| spirohydantoin mustard | 172112 |
| chlorozotocin | 178248 |
| tamoxifen | 180973 |
| AZQ | 182986 |
| spirogermanium | 192965 |
| aclacinomycin A | 208734 |
| 2′-deoxycoformycin | 218321 |
| PALA | 224131 |
| rapamycin | 226080 |
| largomycin | 237020 |
| CBDCA (carboplatin) | 241240 |
| m-AMSA (amsacrine) | 249992 |
| caracemide | 253272 |
| CHIP | 256927 |
| 3-deazaguanine | 261726 |
| dihydro-5-azacytidine | 264880 |
| glycoxalic acid | 267213 |
| deoxydoxorubicin | 267469 |
| N,N-dibenzyldaunomycin | 268242 |
| menogaril | 269148 |
| (carboxyphthalato)platinum | 271674 |
| pyrrolizine dicarbamate | 278214 |
| triciribine phosphate | 280594 |
| ARA AC | 281272 |
| trimethyltrimethylolmelamine | 283162 |
| mitindomide | 284356 |
| 8Cl-cyc-AMP | 284751 |
| tiazofurin | 286193 |
| pyrimidine-5-glycodialdehyde | 291643 |
| flavoneacetic acid ester | 293015 |
| teroxirone | 296934 |
| DHAD (mitoxantrone) | 301739 |
| aphidicolin glycinate | 303812 |
| L-cysteine analogue | 303861 |
| acodazole hydrochloride | 305884 |
| amonafide | 308847 |
| fludarabine phosphate | 312887 |
| SR2555 (nitroimidazole) | 314055 |
| batracylin | 320846 |
| nitroestrone | 321803 |
| pibenzimol hydrochloride | 322921 |
| bactobolin | 325014 |
| didemnin B | 325319 |
| L-buthionine sulfoximine | 326231 |
| phyllanthoside | 328426 |
| hepsulfam | 329680 |
| macbecin II | 330500 |
| rhizoxin | 332598 |
| tetrocarcin A sodium salt | 333856 |
| merbarone | 336628 |
| bisantrene hydrochloride | 337766 |
| penclomedine | 338720 |
| clomesone | 338947 |
| chloroquinoxaline sulfonamide | 339004 |
| bryostatin 1 | 339555 |
| fostriecin | 339638 |
| dihydrolenperone | 343513 |
| piperazine alkylator | 344007 |
| flavoneacetic acid | 347512 |
| cyclodisone | 348948 |
| pancratiastatin | 349156 |
| oxanthrazole | 349174 |
| 4-ipomeanol | 349438 |
| trimetrexate | 352122 |
| mitozolamide | 353451 |
| morpholino-ADR | 354646 |
| anthrapyrazole | 355644 |
| deoxyspergualin | 356894 |
| cyanomorpholino-ADR | 357704 |
| pyrazine diazohydroxide | 361456 |
| tetraplatin | 363812 |
| pyrazoloacridine | 366140 |
| bispyridocarbazolium DMS | 366241 |
| DUP785 (brequinar) | 368390 |
| cyclopentenylcytosine | 375575 |
| ARA-6-MP | 406021 |
| BCNU | 409962 |
| echinomycin | 526417 |
| carmethizole | 602668 |
| topotecan | 609699 |
| MX2 HCl | 619003 |

### Appendix B

### Developmental Therapeutics Program NCI/NIH

### dtp.nci.nih.gov/branches/btb/ivclsp.html

### Screening Services

### DTP Human Tumor Cell Line Screen

Process

The In Vitro Cell Line Screening Project (IVCLSP) is a dedicated service providing direct support to the DTP anticancer drug discovery program. The in vitro cell line screen was implemented in fully operational form in April of 1990. It required approximately five years (1985-1990) to develop, and persistence in the effort reflected dissatisfaction with the performance of prior in vivo primary screens. This project is designed to screen up to 20,000 compounds per year for potential anticancer activity. The operation of this screen utilizes 60 different human tumor cell lines, representing leukemia, melanoma and cancers of the lung, colon, brain, ovary, breast, prostate, and kidney. The aim is to prioritize for further evaluation, synthetic compounds or natural product samples showing selective growth inhibition or cell killing of particular tumor cell lines. This screen is unique in that the complexity of a 60 cell line dose response produced by a given compound results in a biological response pattern which can be utilized in pattern recognition algorithms. Using these algorithms, it is possible to assign a putative mechanism of action to a test compound, or to determine that the response pattern is unique and not similar to that of any of the standard prototype compounds included in the NCI database (see DTP Overview tab). In addition, following characterization of various cellular molecular targets in the 60 cell lines, it may be possible to select compounds most likely to interact with a specific molecular target.

Methodology Of The In Vitro Cancer Screen

The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO2, 95% air and 100% relative humidity for 24 h prior to addition of experimental drugs.

After 24 h, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO2, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:
[(Ti−Tz)/(C−Tz)]×100 for concentrations for which Ti>/=Tz
[(Ti−Tz)/Tz]×100 for concentrations for which Ti<Tz.

Three dose response parameters are calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC50 (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

### PUBLICATIONS

Alley, M. C., Scudiero, D. A., Monks, P. A., Hursey, M. L., Czerwinski, M. J., Fine, D. L., Abbott, B. J., Mayo, J. G., Shoemaker, R. H., and Boyd, M. R. Feasibility of Drug Screening with Panels of Human Tumor Cell Lines Using a Microculture Tetrazolium Assay. Cancer Research 48: 589-601, 1988.
Grever, M. R., Schepartz, S. A., and Chabner, B. A. The National Cancer Institute: Cancer Drug Discovery and Development Program. Seminars in Oncology, Vol. 19, No. 6, pp 622-638, 1992.
Boyd, M. R., and Paull, K. D. Some Practical Considerations and Applications of the National Cancer Institute In Vitro Anticancer Drug Discovery Screen. Drug Development Research 34: 91-109, 1995.
   Three Cell Line Prescreen

The three cell line, one-dose prescreen identifies a large proportion of the compounds that would be inactive in multi-dose 60 cell line screening. Computer modeling indicates that approximately 50% of compounds can be eliminated by this prescreen without a significant decrease in the ability to identify active agents, while increasing the throughput and efficiency of the main cancer screen with limited loss of information. The current assay utilizes a 384 well plate format and fluorescent staining technologies resulting in greater screening capacity for testing of synthetic samples.

Cell Lines

The cell lines are grown in the same manner as for the 60 cell line screen (see above). The cells are plated a densities of 5000 cells/well (MCF7), 1000 cells/well (NCI-H460), and 7500 cells/well (SF-268) to allow for varying doubling time of the cell lines. Each plate contains all three cell lines, a series of dilutions of standard agents, total kill wells and appropriate controls. Plates are incubated under standard conditions for 24 hours prior to addition of experimental compounds or extracts.

Addition of Experimental Agents (Pure Compounds)

Experimental compounds are solubilized in dimethyl sulfoxide (DMSO) at 400-times the desired maximum test concentration (maximum final DMSO concentration of 0.25%) and stored frozen. Compounds are then diluted with complete media with 0.1% gentamicin sulfate (5 μl of test sample in 100% DMSO is added to 565 μl of complete medium). 20 μl of this solution is then dispensed into test wells containing 50 μl of cell suspension to yield a test concentration of 1.00E-04M.

Two standard drugs, meaning that their activities against the cell lines are well documented, are tested against each cell line: NSC 19893 (5-FU) and NSC 123127 (Adriamycin).

Endpoint Measurement

After compound addition, plates are incubated at standard conditions for 48 hours, 10 μl/well Alamar Blue is added and the plates are incubated for an additional 4 hours. Fluorescence is measured using an excitation wavelength of 530 nm and an emission wavelength of 590 nm.

Calculation of Percent Test Cell Growth/Control (untreated) Cell Growth (T/C)

Percent growth is calculated on a plate-by-plate basis for test wells relative to control wells. Percent Growth is expressed as the ratio of fluorescence of the test well to the average fluorescence of the control wells * 100.

Criteria for Activity

Compounds which inhibit the growth of any of the 3 cell lines to 32% or less than control growth are automatically forwarded for testing in the 60 cell line assay. To validate the selection of 32% as the cutoff point for activity, 208 compounds that produced T/Cs of 32% to 50% in any one cell line in the 3 cell line assay were forwarded to the 60 cell line assay. Of those 208, 17% were considered sufficiently active to warrant a confirmatory 60 cell line experiment. Six percent of the 208 demonstrated confirmed activity upon retest in the 60 cell line screen and were reviewed for possible in vivo testing. Less than 1% of the original 208 were actually selected for follow-up in vivo hollow fiber testing.

Modifications to Screen for Natural Product Extracts

Cell Lines

Cells are harvested as above and plated onto a 96-Well flat-bottom, polystyrene plate in 180 μl standard RPMI -1640 media, at densities of 10,000 cells/well (MCF7), 7500 cells/well (NCI-H460), and 15,000 cells/well (SF-268). Each cell line is plated on duplicate plates: one time-zero plate, and one drug background plate is made with media only added.

Addition of Extracts

Extracts are prepared in DMSO at 400-times the desired maximum test concentration and stored frozen. Extracts are diluted in complete media with 0. 1% Gentamicin sulfate and dispensed into wells in a volume of 20 μl to yield a test concentration of 100 μg/ml. NSC 123127 (Adriamycin) is used as the standard and is included on each plate.

Endpoint Measurement

Cells are fixed in situ by the addition of cold TCA (final concentration 10% TCA) and incubated for 60 minutes at 4° C. The supernatant is discarded, plates washed five times with tap water and air-dried. SRB at a 0.4% (w/v) in 1% acetic acid is added to each well and the plates are incubated for 10 minutes at room temperature. Unbound dye is removed by washing six times with 1% acetic acid and the plates are air-dried. Bound SRB is solubilized with 10 mM trizma base and the absorbance is measured at a wavelength of 515 mm.

Calculation of Percent T/C

Percent growth is calculated from six control wells, time zero wells and one test well for each cell line. % Growth is calculated by the same method used in the 60-cell line primary screen.

| Cell Line | Tumor Type |
|---|---|
| MCF7 | Breast |
| NCI-H460 | Lung |
| SF-268 | CNS |

APPENDIX A
Cell lines in the NCI60 set
dtp.nci.nih.gov/docs/misc/common_files/cell_list.html

| | | Doubling | Inoculation |
|---|---|---|---|
| Cell Line Name | Panel Name | Time | Density |
| CCRF-CEM | Leukemia | 26.7 | 40000 |
| HL-60(TB) | Leukemia | 28.6 | 40000 |
| K-562 | Leukemia | 19.6 | 5000 |
| MOLT-4 | Leukemia | 27.9 | 30000 |
| RPMI-8226 | Leukemia | 33.5 | 20000 |
| SR | Leukemia | 28.7 | 20000 |
| A549/ATCC | Non-Small Cell Lung | 22.9 | 7500 |
| EKVX | Non-Small Cell Lung | 43.6 | 20000 |
| HOP-62 | Non-Small Cell Lung | 39 | 10000 |
| HOP-92 | Non-Small Cell Lung | 79.5 | 20000 |
| NCI-H226 | Non-Small Cell Lung | 61 | 20000 |
| NCI-H23 | Non-Small Cell Lung | 33.4 | 20000 |
| NCI-H322M | Non-Small Cell Lung | 35.3 | 20000 |
| NCI-H460 | Non-Small Cell Lung | 17.8 | 7500 |
| NCI-H522 | Non-Small Cell Lung | 38.2 | 20000 |
| COLO 205 | Colon | 23.8 | 15000 |
| HCC-2998 | Colon | 31.5 | 15000 |
| HCT-116 | Colon | 17.4 | 5000 |
| HCT-15 | Colon | 20.6 | 10000 |
| HT29 | Colon | 19.5 | 5000 |
| KM12 | Colon | 23.7 | 15000 |
| SW-620 | Colon | 20.4 | 10000 |
| SF-268 | CNS | 33.1 | 15000 |
| SF-295 | CNS | 29.5 | 10000 |
| SF-539 | CNS | 35.4 | 15000 |
| SNB-19 | CNS | 34.6 | 15000 |
| SNB-75 | CNS | 62.8 | 20000 |
| U251 | CNS | 23.8 | 7500 |
| LOX IMVI | Melanoma | 20.5 | 7500 |
| MALME-3M | Melanoma | 46.2 | 20000 |
| M14 | Melanoma | 26.3 | 15000 |
| SK-MEL-2 | Melanoma | 45.5 | 20000 |
| SK-MEL-28 | Melanoma | 35.1 | 10000 |
| SK-MEL-5 | Melanoma | 25.2 | 10000 |
| UACC-257 | Melanoma | 38.5 | 20000 |
| UACC-62 | Melanoma | 31.3 | 10000 |
| IGR-OV1 | Ovarian | 31 | 10000 |
| OVCAR-3 | Ovarian | 34.7 | 10000 |
| OVCAR-4 | Ovarian | 41.4 | 15000 |
| OVCAR-5 | Ovarian | 48.8 | 20000 |
| OVCAR-8 | Ovarian | 26.1 | 10000 |
| SK-OV-3 | Ovarian | 48.7 | 20000 |
| 786-0 | Renal | 22.4 | 10000 |
| A498 | Renal | 66.8 | 25000 |
| ACHN | Renal | 27.5 | 10000 |
| CAKI-1 | Renal | 39 | 10000 |
| RXF 393 | Renal | 62.9 | 15000 |
| SN12C | Renal | 29.5 | 15000 |
| TK-10 | Renal | 51.3 | 15000 |
| UO-31 | Renal | 41.7 | 15000 |
| PC-3 | Prostate | 27.1 | 7500 |
| DU-145 | Prostate | 32.3 | 10000 |
| MCF7 | Breast | 25.4 | 10000 |
| NCI/ADR-RES | Breast | 34 | 15000 |
| MDA-MB-231/ATCC | Breast | 41.9 | 20000 |
| HS 578T | Breast | 53.8 | 20000 |
| MDA-MB-435 | Breast | 25.8 | 15000 |
| BT-549 | Breast | 53.9 | 20000 |
| T-47D | Breast | 45.5 | 20000 |

1. A method of classifying a tumor as likely or unlikely to respond to therapy with a chemical compound comprising steps of:
providing a tumor sample from a patient;
detecting in the tumor sample a correlated biomarker characterized in that presence or absence of the biomarker has been correlated with responsiveness or lack of responsiveness to a selected chemical compound; and
classifying the tumor as likely or unlikely to respond to therapy with the selected chemical compound based on the results of the detection step.

2. The method of claim 1, wherein the step of detecting comprises:
contacting the tumor sample with an antibody that binds specifically to the correlated biomarker; and
detecting binding by the antibody to the tumor sample.

3. The method of claim 1, wherein the step of detecting comprises:
contacting the tumor sample with a nucleotide probe that binds specifically to a product of the correlated biomarker's gene; and
detecting binding by the nucleotide probe to the tumor sample.

4. The method of claim 1, wherein presence or absence of the biomarker has been correlated with responsiveness to the selected chemical compound.

5. The method of claim 1, wherein presence or absence of the biomarker has been correlated with a lack of responsiveness to the selected chemical compound.

6. The method of claim 1, wherein:
the correlated biomarker is selected from the group consisting of Hs.23643, Hs.279949, Hs.3566, and Hs.151903, and the chemical compound is methotrexate; or
the correlated biomarker is Hs.274453, and the chemical compound is 8-azaguanine; or
the correlated biomarker is Hs.24427, and the chemical compound is nitrogen mustard; or
the correlated biomarker is Hs.220594, and the chemical compound is fluorouracil; or
the correlated biomarker is Hs.274453, and the chemical compound is 7-Hydroxy-v-triazolo[d]pyrimidine; or
the correlated biomarker is Hs.5737, and the chemical compound is mytomycin C; or
the correlated biomarker is Hs.274453, and the chemical compound is aquamycin; or
the correlated biomarker is Hs.89433, and the chemical compound is sudan R; or
the correlated biomarker is Hs.76662, and the chemical compound is cytarabine hydrochloride; or
the correlated biomarker is Hs. 198307, and the chemical compound is vincristine sulfate; or
the correlated biomarker is Hs.274453, and the chemical compound is 2-Naphthacenecarboxamide, N-[(6,7-dihydro-7-oxo-v-triazolo[4, 5 d]pyrimidin-5-ylamino)methyl]-4-dimethylamino-1,4,4a,5,5a,6,11,12a-octahydro-3,6,10,12,12a-pentahydroxy-6-methyl-1,11-dioxo-; or
the correlated biomarker is selected from the group consisting of Hs.24427 and Hs.24643, and the chemical compound is daunorubicin hydrochloride; or
the correlated biomarker is Hs.343521, and the chemical compound is tritylcysteine; or
the correlated biomarker is Hs.22549, and the chemical compound is adenosine, 2-chloro-2′-deoxy-; or
the correlated biomarker is Hs.211563, and the chemical compound is inosine dialdehyde; or
the correlated biomarker is Hs.235709, and the chemical compound is cisplatin; or
the correlated biomarker is Hs.24427, and the chemical compound is teniposide; or
the correlated biomarker is Hs.23643, and the chemical compound is methasquin; or
the correlated biomarker is Hs.3321, and the chemical compound is azirino[2′,3′:3,4]pyrrolo[1,2-a]indole-4,7-dione, 1,1a,2,8,8a, 8b-hexahydro-8-(hydroxymethyl)-8a-methoxy-5-methyl-6-(propylamino)-, carbamate (ester); or
the correlated biomarker is selected from the group consisting of Hs.24427 and Hs.343667, and the chemical compound is doxorubicin; or
the correlated biomarker is selected from the group consisting of Hs.318501, Hs.7835, and Hs.182874, and the chemical compound is bleomycin; or
the correlated biomarker is selected from the group consisting of Hs.198307 and Hs.86896, and the chemical compound is paclitaxel; or
the correlated biomarker is selected from the group consisting of Hs.279949 and Hs.151903, and the chemical compound is pyrazofurin; or
the correlated biomarker is Hs.155956, and the chemical compound is maleimide, N-(1-hydroxyacetonyl)-; or
the correlated biomarker is selected from the group consisting of Hs.282093, Hs.321264, Hs.108502, and Hs.692, and the chemical compound is NSC # 150412; or
the correlated biomarker is Hs.93605, and the chemical compound is pyrido[3,2-g]quinoline-2,5,8,10(1H,9H)-tetrone, 1,4,6-trimethyl-; or
the correlated biomarker is Hs.75909, and the chemical compound is 2,6-piperazinedione, 4,4′-propylenedi-, (P)-; or
the correlated biomarker is Hs. 151903, and the chemical compound is L-Glutamic acid, N-[[4-[[(2,4-diamino-6-pteridinyl)methyl]methylamino]-1-naphthalenyl]carbonyl ]-; or
the correlated biomarker is Hs.350470, and the chemical compound is tamoxifen; or
the correlated biomarker is Hs.24427, and the chemical compound is largomycin F-II; or
the correlated biomarker is Hs.10784, and the chemical compound is carboplatin; or
the correlated biomarker is Hs.125359, and the chemical compound is benzo[c]phenthridinium, 3-hydroxy-2,8,9-trimethoxy-5-methyl-, chloride; or
the correlated biomarker is Hs. 154672, and the chemical compound is oxaliplatin; or
the correlated biomarker is selected from the group consisting of Hs.24427 and Hs.24643, and the chemical compound is deoxydoxorubicin; or
the correlated biomarker is Hs.125359, and the chemical compound is thalphenine chloride; or
the correlated biomarker is Hs.77432, and the chemical compound is triciribine phosphate; or
the correlated biomarker is Hs.355533, and the chemical compound is adenosine, 8-chloro-, cyclic 3′,5′-(hydrogen phosphate); or
the correlated biomarker is selected from the group consisting of Hs.182874 and Hs.235709, and the chemical compound is mitoxantrone; or
the correlated biomarker is Hs.22549, and the chemical compound is fludarabine phosphate; or
the correlated biomarker is Hs.6682, and the chemical compound is selendale; or
the correlated biomarker is Hs.108301, and the chemical compound is trimetrexate; or
the correlated biomarker is selected from the group consisting of Hs.23643 and Hs.444382, and the chemical compound is pyrazoloacridine; or
the correlated biomarker is Hs.23643, and the chemical compound is biphenquinate; or
the correlated biomarker is Hs.1 1465, and the chemical compound is acronycine, 2-nitro; or
the correlated biomarker is selected from the group consisting of Hs.274428 and Hs.235709, and the chemical compound is hycamtin; or
the correlated biomarker is Hs.235709, and the chemical compound is gemcitabine; or
the correlated biomarker is Hs.24427, and the chemical compound is irinotecan; or
the correlated biomarker is Hs.274453, and the chemical compound is benzo[1,2-c:4,5-c′]dipyrrole-1,3,5,7(2H,6H)-tetraimine; or
the correlated biomarker is Hs.75789, and the chemical compound is ferprenin; or
the correlated biomarker is Hs.302740, and the chemical compound is NSC # 627745; or
the correlated biomarker is Hs.29276, and the chemical compound is taxotere; or
the correlated biomarker is Hs.235782, and the chemical compound is perifosine; or
the correlated biomarker is Hs.6682, and the chemical compound is NSC # 641171; or
the correlated biomarker is Hs. 182575, and the chemical compound is NSC # 647133; or
the correlated biomarker is Hs.389700, and the chemical compound is halomon; or
the correlated biomarker is Hs.93605, and the chemical compound is NSC # 656238; or
the correlated biomarker is Hs.24427, and the chemical compound is 6H-pyrido[4,3-b]carbazole-1-carboxamide, 9-hydroxy-5,6-dimethyl-N-[2-(dimethylamino)ethyl]-, dihydrochloride; or
the correlated biomarker is selected from the group consisting of Hs.184601 and Hs.79748, and the chemical compound is kahalalide F; or
the correlated biomarker is Hs.198689, and the chemical compound is NSC # 672293;or
the correlated biomarker is Hs.389700, and the chemical compound is (+)-6-bromo-3-bromomethyl-2,3,7-trichloro-7-methyl-1-octene; or
the correlated biomarker is Hs.6682, and the chemical compound is NSC # 673997; or
the correlated biomarker is Hs.274453, and the chemical compound is 5-hydroxy-7-amino(1,2,3)thiadiazole[5,4-d]pyrimidine; or
the correlated biomarker is selected from the group consisting of Hs. 89433, Hs.77432, and Hs.75216, and the chemical compound is NSC # 676495; or
the correlated biomarker is Hs.274453, and the chemical compound is 7H-5,6-dithioleno[4,3-d]uracil; or
the correlated biomarker is Hs.63609, and the chemical compound is 1-methyl-3-(4-[2-dimethylaminoethoxy]phenyl)-2-phenylindolizine; or
the correlated biomarker is Hs. 155956, and the chemical compound is carbamic acid, [3-[4-[bis(2-chloroethyl)amino]phenyl]propyl]-, 2-[[6-[5-hydroxy-2-(4-hydroxyphenyl)-3-methyl-1H-indol-1-yl]hexyl]amino]ethyl ester; or
the correlated biomarker is Hs. 198689, and the chemical compound is NSC # 697032; or
the correlated biomarker is Hs.82568, and the chemical compound is 1,6-bis[4-(4-aminophenoxy)phenyl]diamantine; or
the correlated biomarker is Hs.6682, and the chemical compound is NSC # 708980; or
the correlated biomarker is selected from the group consisting of Hs.82961 and Hs.350470, and the chemical compound is bryodulcosigenin; or
the correlated biomarker is Hs.63609, and the chemical compound is NSC # 715524; or
the correlated biomarker is Hs.21330, and the chemical compound is dactinomycin; or
the correlated biomarker is Hs. 100469, and the chemical compound is melphalan; or
the correlated biomarker is selected from the group consisting of Hs.277401 and Hs.409080, and the chemical compound is vinblastine sulfate; or
the correlated biomarker is Hs.21330, and the chemical compound is daunorubicin hydrochloride; or
the correlated biomarker is Hs.76550, and the chemical compound is urea, 1-(2-chloroethyl)-3-(2,6-dioxo-3-piperidyl)-1-nitroso-; or
the correlated biomarker is selected from the group consisting of Hs.68879, Hs.85266, and Hs.399815, and the chemical compound is cisplatin; or
the correlated biomarker is Hs.2399, and the chemical compound is teniposide; or
the correlated biomarker is Hs.59255, and the chemical compound is bleomycin; or
the correlated biomarker is Hs.21330, and the chemical compound is paclitaxel; or
the correlated biomarker is selected from the group consisting of Hs.426222 and Hs.79172, and the chemical compound is tamoxifen; or
the correlated biomarker is Hs.85266, and the chemical compound is carboplatin; or
the correlated biomarker is Hs.2399, and the chemical compound is celiptium; or
the correlated biomarker is Hs.59255, and the chemical compound is mitoxantrone; or
the correlated biomarker is Hs.424212, and the chemical compound is perifosine; or
the correlated biomarker is selected from the group consisting of Hs.6682 and Hs. 151393, and the chemical compound is adophostin.

7. A method of identifying biomarkers that are predictive of tumor responsiveness to particular chemical compounds, the method comprising steps of:
providing an expression or activity dataset for a predetermined collection of tumor cells;
providing a chemical compound toxicity dataset for the tumor cells; and
establishing a correlation between expression of at least one biomarker and toxicity of at least one compound such that expression of the at least one biomarker is predictive of tumor responsiveness to the at least one compound.

8. A method of identifying chemical compounds whose ability to inhibit tumor cell growth correlates with expression of a particular biomarker, the method comprising steps of:
providing an expression or activity dataset for a predetermined collection of tumor cells;
providing a chemical compound toxicity dataset for the tumor cells; and
establishing a correlation between expression of at least one biomarker and toxicity of at least one compound such that the compound is predicted to effectively inhibit growth of tumor cells expressing the biomarker.

9. The method of claim 7 or 8, wherein a positive correlation between expression of the at least one biomarker and toxicity of the at least one compound is established.

10. The method of claim 7 or 8, wherein a negative correlation between expression of the at least one biomarker and toxicity of the at least one compound is established.

11. The method of claim 7 or 8, further comprising a step of filtering the expression or activity dataset or filtering the chemical compound toxicity dataset before the step of establishing a correlation.

12. The method of claim 11, wherein the expression or activity dataset is filtered by excluding data points selected from the group consisting of:
data points from genes that are undetectable in a meaningful fraction;
data points from leukemia cell lines;
data points for which kurtosis is too high;
data points reflecting broad physiological characteristics; and
data points that show insignificant variance.

13. The method of claim 11, wherein the chemical compound toxicity dataset is filtered by excluding data points selected from the group consisting of:
data points for compounds for which measurements are unavailable for a significant fraction of the collection of tumor cells;
data points for compounds that show little or no toxicity across the collection of tumor cells;
data points for compounds with toxicity that varied minimally around a large concentration;
data points for which the spread of data is less than the average variation found within repeat measurements of the cytotoxicity within each cell line for a given compound; and
data points for which kurtosis is too high.

14. The method of claim 7 or 8, further comprising a step of assessing the quality of the established correlation by:
comparing correlations where certain cell lines are removed;
requiring that a minimum number of cell lines contribute to the correlation; or
excluding correlations with compounds for which the kurtosis in the GI50 is too high.

15. The method of claim 7 or 8, wherein a plurality of biomarker:compound correlates are established, the method further comprising a step of selecting pairs that satisfy one or more selection rules selected from the group consisting of:
the biomarker expression and compound cytotoxicity having a minimal correlation strength;
the biomarker displaying a desired pattern of expression across normal and tumor tissues;
the compound having a minimal potency as measured by the GI50 assessment of cytotoxicity;
the biomarker having a minimal level of expression;
the biomarker having prior information regarding its potential to serve as a pharmacological target for cancer;
the compound having prior information regarding its applicability for pharmacological modification for drugability;
the compound GI50 pattern being unique amongst the measured compounds in the cytotoxicity dataset; and
the biomarker expression or activity pattern being unique amongst the expression or activity dataset.

16. The method of claim 7 or 8, wherein the at least one compound is a small molecule.

17. The method of claim 7 or 8, wherein the at least one compound is an antibody or other specific ligand of the at least one biomarker.

18. A method of treating cancer comprising:
detecting in a tumor sample from a patient a correlated biomarker selected from the group consisting of Hs.23643, Hs.279949, Hs.3566, and Hs.151903; and
administering to the patient a therapeutic agent that comprises methotrexate; or detecting in a tumor sample from a patient the correlated biomarker Hs.274453; and
administering to the patient a therapeutic agent that comprises 8-azaguanine; or detecting in a tumor sample from a patient the correlated biomarker Hs.24427; and
administering to the patient a therapeutic agent that comprises nitrogen mustard; or
detecting in a tumor sample from a patient the correlated biomarker Hs.220594; and
administering to the patient a therapeutic agent that comprises fluorouracil; or
detecting in a tumor sample from a patient the correlated biomarker Hs.274453; and
administering to the patient a therapeutic agent that comprises 7-hydroxy-v-triazolo[d]pyrimidine; or
detecting in a tumor sample from a patient the correlated biomarker Hs.5737; and
administering to the patient a therapeutic agent that comprises mytomycin C; or
detecting in a tumor sample from a patient the correlated biomarker Hs.274453; and
administering to the patient a therapeutic agent that comprises aquamycin; or
detecting in a tumor sample from a patient the correlated biomarker Hs.89433; and
administering to the patient a therapeutic agent that comprises sudan R; or
detecting in a tumor sample from a patient the correlated biomarker Hs.76662; and
administering to the patient a therapeutic agent that comprises cytarabine hydrochloride; or
detecting in a tumor sample from a patient the correlated biomarker Hs.198307; and
administering to the patient a therapeutic agent that comprises vincristine sulfate; or
detecting in a tumor sample from a patient the correlated biomarker Hs.274453; and
administering to the patient a therapeutic agent that comprises 2-naphthacenecarboxamide, N-[(6,7-dihydro-7-oxo-v-triazolo[4,5d]pyrimidin-5-ylamino)methyl]-4-dimethylamino-1,4,4a,5,5a,6,11,12a-octahydro-3,6,10,12,12a-pentahydroxy-6-methyl-1,11-dioxo-; or
detecting in a tumor sample from a patient a correlated biomarker selected from the group consisting of Hs.24427 and Hs.24643; and
administering to the patient a therapeutic agent that comprises daunorubicin hydrochloride; or
detecting in a tumor sample from a patient the correlated biomarker Hs.343521; and
administering to the patient a therapeutic agent that comprises tritylcysteine; or
detecting in a tumor sample from a patient the correlated biomarker Hs.22549; and
administering to the patient a therapeutic agent that comprises adenosine, 2-chloro-2′-deoxy-; or
detecting in a tumor sample from a patient the correlated biomarker Hs.211563; and
administering to the patient a therapeutic agent that comprises inosine dialdehyde; or
detecting in a tumor sample from a patient the correlated biomarker Hs.235709; and
administering to the patient a therapeutic agent that comprises cisplatin; or
detecting in a tumor sample from a patient the correlated biomarker Hs.24427; and
administering to the patient a therapeutic agent that comprises teniposide; or
detecting in a tumor sample from a patient the correlated biomarker Hs.23643; and
administering to the patient a therapeutic agent that comprises methasquin; or
detecting in a tumor sample from a patient the correlated biomarker Hs.3321; and
administering to the patient a therapeutic agent that comprises azirino[2′,3:3,4]pyrrolo[1,2-a]indole-4,7-dione, 1,1a,2,8,8a, 8b-hexahydro-8-(hydroxymethyl)-8a-methoxy-5-methyl-6-(propylamino)-, carbamate (ester); or
detecting in a tumor sample from a patient a correlated biomarker selected from the group consisting of Hs.24427 and Hs.343667; and
administering to the patient a therapeutic agent that comprises doxorubicin; or
detecting in a tumor sample from a patient a correlated biomarker selected from the group consisting of Hs.318501, Hs.7835, and Hs.182874; and
administering to the patient a therapeutic agent that comprises bleomycin; or
detecting in a tumor sample from a patient a correlated biomarker selected from the group consisting of Hs.198307 and Hs.86896; and
administering to the patient a therapeutic agent that comprises paclitaxel; or
detecting in a tumor sample from a patient a correlated biomarker selected from the group consisting of Hs.279949 and Hs.151903; and
administering to the patient a therapeutic agent that comprises pyrazofurin; or
detecting in a tumor sample from a patient the correlated biomarker Hs.155956; and
administering to the patient a therapeutic agent that comprises maleimide, N-(1-hydroxyacetonyl)-; or
detecting in a tumor sample from a patient a correlated biomarker selected from the group consisting of Hs.282093, Hs.321264, Hs.108502, and Hs.692; and
administering to the patient a therapeutic agent that comprises NSC # 150412; or
detecting in a tumor sample from a patient the correlated biomarker Hs.93605; and
administering to the patient a therapeutic agent that comprises pyrido[3,2-g]quinoline-2,5,8,10(1H,9H)-tetrone, 1,4,6-trimethyl-; or
detecting in a tumor sample from a patient the correlated biomarker Hs.75909; and
administering to the patient a therapeutic agent that comprises 2,6-piperazinedione, 4,4′-propylenedi-, (P)-; or
detecting in a tumor sample from a patient the correlated biomarker Hs.151903; and
administering to the patient a therapeutic agent that comprises L-glutamic acid, N-[[4-[[(2,4-diamino-6-pteridinyl)methyl]methylamino]-1-naphthalenyl]carbonyl]-; or
detecting in a tumor sample from a patient the correlated biomarker Hs.350470; and
administering to the patient a therapeutic agent that comprises tamoxifen; or
detecting in a tumor sample from a patient the correlated biomarker Hs.24427; and
administering to the patient a therapeutic agent that comprises largomycin F-II; or
detecting in a tumor sample from a patient the correlated biomarker Hs. 10784; and
administering to the patient a therapeutic agent that comprises carboplatin; or
detecting in a tumor sample from a patient the correlated biomarker Hs.125359; and
administering to the patient a therapeutic agent that comprises benzo[c]phenthridinium, 3-hydroxy-2,8,9-trimethoxy-5-methyl-, chloride; or
detecting in a tumor sample from a patient the correlated biomarker Hs. 154672; and
administering to the patient a therapeutic agent that comprises oxaliplatin; or
detecting in a tumor sample from a patient a correlated biomarker selected from the group consisting of Hs.24427 and Hs.24643; and
administering to the patient a therapeutic agent that comprises deoxydoxorubicin;or
detecting in a tumor sample from a patient the correlated biomarker Hs.125359; and
administering to the patient a therapeutic agent that comprises thalphenine chloride; or
detecting in a tumor sample from a patient the correlated biomarker Hs.77432; and
administering to the patient a therapeutic agent that comprises triciribine phosphate; or
detecting in a tumor sample from a patient the correlated biomarker Hs.355533; and
administering to the patient a therapeutic agent that comprises Adenosine, 8-chloro-, cyclic 3′,5′-(hydrogen phosphate); or
detecting in a tumor sample from a patient a correlated biomarker selected from the group consisting of Hs.182874 and Hs.235709; and
administering to the patient a therapeutic agent that comprises mitoxantrone; or
detecting in a tumor sample from a patient the correlated biomarker Hs.22549; and
administering to the patient a therapeutic agent that comprises fludarabine phosphate; or
detecting in a tumor sample from a patient the correlated biomarker Hs.6682; and
administering to the patient a therapeutic agent that comprises selendale; or
detecting in a tumor sample from a patient the correlated biomarker Hs. 108301; and
administering to the patient a therapeutic agent that comprises trimetrexate; or
detecting in a tumor sample from a patient a correlated biomarker selected from the group consisting of Hs.23643 and Hs.444382; and
administering to the patient a therapeutic agent that comprises pyrazoloacridine; or
detecting in a tumor sample from a patient the correlated biomarker Hs.23643; and
administering to the patient a therapeutic agent that comprises biphenquinate; or
detecting in a tumor sample from a patient the correlated biomarker Hs.11465; and
administering to the patient a therapeutic agent that comprises acronycine, 2-nitro; or
detecting in a tumor sample from a patient a correlated biomarker selected from the group consisting of Hs.274428 and Hs.235709; and
administering to the patient a therapeutic agent that comprises hycamtin; or
detecting in a tumor sample from a patient the correlated biomarker Hs.235709; and
administering to the patient a therapeutic agent that comprises gemcitabine; or
detecting in a tumor sample from a patient the correlated biomarker Hs.24427; and
administering to the patient a therapeutic agent that comprises irinotecan; or
detecting in a tumor sample from a patient the correlated biomarker Hs.274453; and
administering to the patient a therapeutic agent that comprises benzo[1,2-c:4,5-c′]dipyrrole-1,3,5,7(2H,6H)-tetraimine; or
detecting in a tumor sample from a patient the correlated biomarker Hs.75789; and
administering to the patient a therapeutic agent that comprises ferprenin; or
detecting in a tumor sample from a patient the correlated biomarker Hs.302740; and
administering to the patient a therapeutic agent that comprises NSC # 627745; or
detecting in a tumor sample from a patient the correlated biomarker Hs.29276; and
administering to the patient a therapeutic agent that comprises taxotere; or
detecting in a tumor sample from a patient the correlated biomarker Hs.235782; and
administering to the patient a therapeutic agent that comprises perifosine; or
detecting in a tumor sample from a patient the correlated biomarker Hs.6682; and
administering to the patient a therapeutic agent that comprises NSC # 641171; or
detecting in a tumor sample from a patient the correlated biomarker Hs.182575; and
administering to the patient a therapeutic agent that comprises NSC # 647133; or
detecting in a tumor sample from a patient the correlated biomarker Hs.389700; and
administering to the patient a therapeutic agent that comprises halomon; or
detecting in a tumor sample from a patient the correlated biomarker Hs.93605; and
administering to the patient a therapeutic agent that comprises NSC # 656238; or
detecting in a tumor sample from a patient the correlated biomarker Hs.24427; and
administering to the patient a therapeutic agent that comprises 6H-pyrido[4,3-b]carbazole -1-carboxamide, 9-hydroxy-5,6-dimethyl-N-[2-(dimethylamino)ethyl]-, dihydrochloride; or
detecting in a tumor sample from a patient a correlated biomarker selected from the group consisting of Hs.184601 and Hs.79748; and
administering to the patient a therapeutic agent that comprises kahalalide F; or
detecting in a tumor sample from a patient the correlated biomarker Hs.198689; and
administering to the patient a therapeutic agent that comprises NSC # 672293; or
detecting in a tumor sample from a patient the correlated biomarker Hs.389700; and
administering to the patient a therapeutic agent that comprises (+)-6-bromo-3-bromomethyl-2,3,7-trichloro-7-methyl-1-octene; or
detecting in a tumor sample from a patient the correlated biomarker Hs.6682; and
administering to the patient a therapeutic agent that comprises NSC # 673997; or
detecting in a tumor sample from a patient the correlated biomarker Hs.274453; and
administering to the patient a therapeutic agent that comprises 5-hydroxy-7-amino(1,2,3)thiadiazole[5,4-d]pyrimidine; or
detecting in a tumor sample from a patient a correlated biomarker selected from the group consisting of Hs.89433, Hs.77432, and Hs.75216; and
administering to the patient a therapeutic agent that comprises NSC # 676495; or
detecting in a tumor sample from a patient the correlated biomarker Hs.274453; and
administering to the patient a therapeutic agent that comprises 7H-5,6-dithioleno[4,3-d]uracil; or
detecting in a tumor sample from a patient the correlated biomarker Hs.63609; and
administering to the patient a therapeutic agent that comprises 1-methyl-3-(4-[2-dimethylaminothoxy]phenyl)-2-phenylindolizine; or
detecting in a tumor sample from a patient the correlated biomarker Hs.155956; and
administering to the patient a therapeutic agent that comprises carbamic acid, [3-[4-[bis(2-chloroethyl)amino]phenyl]propyl]-, 2-[[6-[5-hydroxy-2-(4-hydroxyphenyl)-3-methyl-1H-indol-1-yl]hexyl]amino]ethyl ester; or
detecting in a tumor sample from a patient the correlated biomarker Hs.198689; and
administering to the patient a therapeutic agent that comprises NSC # 697032; or
detecting in a tumor sample from a patient the correlated biomarker Hs.82568; and
administering to the patient a therapeutic agent that comprises 1,6-bis[4-(4-aminophenoxy)phenyl]diamantine; or
detecting in a tumor sample from a patient the correlated biomarker Hs.6682; and
administering to the patient a therapeutic agent that comprises NSC # 708980; or
detecting in a tumor sample from a patient a correlated biomarker selected from the group consisting of Hs.82961 and Hs.350470; and
administering to the patient a therapeutic agent that comprises bryodulcosigenin; or
detecting in a tumor sample from a patient the correlated biomarker Hs.63609; and
administering to the patient a therapeutic agent that comprises NSC # 715524.

19. A method of treating cancer comprising:
detecting in a tumor sample from a patient the correlated biomarker Hs.274453; and
administering to the patient a therapeutic agent that comprises a compound selected from the group consisting of 8-azaguanine, 7-hydroxy-v-triazolo[d]pyrimidine, and aquamycin; or
detecting in a tumor sample from a patient the correlated biomarker Hs.89433; and
administering to the patient a therapeutic agent that comprises sudan R; or
detecting in a tumor sample from a patient the correlated biomarker Hs.274453; and
administering to the patient a therapeutic agent that comprises 2-naphthacenecarboxamide, N-[(6,7-dihydro-7-oxo-v-triazolo[4,5d]pyrimidin-5-ylamino)methyl]-4-dimethylamino-1,4,4a,5,5 a,6,11,12a-octahydro-3,6,10,12,12a-pentahydroxy-6-methyl-1,11-dioxo-; or
detecting in a tumor sample from a patient the correlated biomarker Hs.155956; and
administering to the patient a therapeutic agent that comprises maleimide, N-(1-hydroxyacetonyl)-; or
detecting in a tumor sample from a patient a correlated biomarker selected from the group consisting of Hs.282093, Hs.321264, Hs.108502, and Hs.692; and
administering to the patient a therapeutic agent that comprises NSC # 150412; or
detecting in a tumor sample from a patient the correlated biomarker Hs.93605; and
administering to the patient a therapeutic agent that comprises pyrido[3,2-g]quinoline-2,5,8,10(1H,9H)-tetrone, 1,4,6-trimethyl-; or
detecting in a tumor sample from a patient the correlated biomarker Hs.125359; and
administering to the patient a therapeutic agent that comprises a compound selected from the group consisting of benzo[c]phenthridinium, 3-hydroxy-2,8,9-trimethoxy-5-methyl-, chloride and thalphenine chloride; or
detecting in a tumor sample from a patient the correlated biomarker Hs.11465; and
administering to the patient a therapeutic agent that comprises acronycine, 2-nitro; or
detecting in a tumor sample from a patient the correlated biomarker Hs.274453; and
administering to the patient a therapeutic agent that comprises benzo[1,2-c:4,5-c′]dipyrrole-1,3,5,7(2H,6H)-tetraimine; or
detecting in a tumor sample from a patient the correlated biomarker Hs.302740; and
administering to the patient a therapeutic agent that comprises NSC # 627745; or
detecting in a tumor sample from a patient the correlated biomarker Hs.6682; and
administering to the patient a therapeutic agent that comprises NSC # 641171; or
detecting in a tumor sample from a patient the correlated biomarker Hs.182575; and
administering to the patient a therapeutic agent that comprises NSC # 647133; or
detecting in a tumor sample from a patient the correlated biomarker Hs.389700; and
administering to the patient a therapeutic agent that comprises halomon; or
detecting in a tumor sample from a patient the correlated biomarker Hs.93605; and
administering to the patient a therapeutic agent that comprises NSC # 656238; or
detecting in a tumor sample from a patient the correlated biomarker Hs.389700; and
administering to the patient a therapeutic agent that comprises (+)-6-bromo-3-bromomethyl-2,3,7-trichloro-7-methyl-1-octene; or
detecting in a tumor sample from a patient the correlated biomarker Hs.6682; and
administering to the patient a therapeutic agent that comprises NSC # 673997; or
detecting in a tumor sample from a patient the correlated biomarker Hs.274453; and
administering to the patient a therapeutic agent that comprises 5-hydroxy-7-amino(1,2,3)thiadiazole[5,4-d]pyrimidine; or
detecting in a tumor sample from a patient a correlated biomarker selected from the group consisting of Hs.89433, Hs.77432, and Hs.75216; and
administering to the patient a therapeutic agent that comprises NSC # 676495; or
detecting in a tumor sample from a patient the correlated biomarker Hs.274453; and
administering to the patient a therapeutic agent that comprises 7H-5,6-dithioleno[4,3-d]uracil; or
detecting in a tumor sample from a patient the correlated biomarker Hs.155956; and
administering to the patient a therapeutic agent that comprises carbamic acid, [3-[4-[bis(2-chloroethyl)amino]phenyl]propyl]-, 2-[[6-[5-hydroxy-2-(4-hydroxyphenyl)-3-methyl-1H-indol-1-yl]hexyl]amino]ethyl ester; or
detecting in a tumor sample from a patient the correlated biomarker Hs.82568; and
administering to the patient a therapeutic agent that comprises 1,6-bis[4-(4-aminophenoxy)phenyl]diamantine; or
detecting in a tumor sample from a patient the correlated biomarker Hs.6682; and
administering to the patient a therapeutic agent that comprises NSC # 708980.


SEQUENCE LISTING (2 sequences)

SEQ ID NO: 1 - DNA, 20 bp, Artificial
  Human cDNA clone primer.
ttgtaaaacgacggccagtg

SEQ ID NO: 2 - DNA, 19 bp, Artificial
  Human cDNA clone primer.
cacacaggaaacagctatg